(19) United States
(12) Patent Application Publication
(10) Pub. No.: US 2026/0366054 A1
(43) Pub. Date: Nov. 19, 2026

(54) METHODS FOR OBTAINING STEVIOL GLYCOSIDES

(71) Applicant: Tate & Lyle Solutions USA LLC, Hoffman Estates, IL (US)

(72) Inventors: Brian Albert, Hoffman Estates, IL (US); Joshua Fletcher, Hoffman Estates, IL (US); Ayano Sakai, Hoffman Estates, IL (US); Timothy Windebank, Hoffman Estates, IL (US); Ryan Gross, Hoffman Estates, IL (US)

(21) Appl. No.: 19/135,659

(22) PCT Filed: Dec. 4, 2023
(86) PCT No.: PCT/IB2023/062203

§ 371 Date: Jun. 4, 2025

Related U.S. Application Data
(60) Provisional application No. 63/430,336, filed on Dec. 5, 2022.

(30) Foreign Application Priority Data
Feb. 20, 2023 (GB) 2302401.1

(51) Int. Cl. C12P 19/56; A23L 2/60; A23L 27/30; C12N 9/10; C12P 19/18

(52) U.S. Cl. CPC C12P 19/56; A23L 2/60; A23L 27/36; C12N 9/1051; C12N 9/1062; C12P 19/18

(57) ABSTRACT

The present disclosure provides methods for obtaining steviol glycosides from stevia material using an enzyme mixture, wherein the stevia material can be an extract, co-product, or waste product from a stevia extraction process involving enzymes β-1,2- and β-1,3-glycosyltransferases and sucrose synthase. Also provided are steviol glycoside compositions that may be prepared using such methods, and uses of said compositions in foods, beverages and the like.

### FIELD OF THE DISCLOSURE

The present disclosure provides methods for obtaining steviol glycosides from stevia material using an enzyme mixture, wherein the stevia material can be an extract, co-product, or waste product from a stevia extraction process. Also provided are steviol glycoside compositions that may be prepared using such methods, and uses of said compositions in foods, beverages and the like.

### BACKGROUND

Steviol glycosides are plant secondary metabolites which have a desirable naturally occurring sweet taste. Sweet steviol glycosides are extracted from Stevia rebaudiana (“Stevia”). These glycosides have commercial value and are used as non-caloric sweeteners. Stevia contains multiple steviol glycosides, but currently only Rebaudioside A and stevioside are broadly commercially available. However, other glycosides extracted from Stevia also have a sweet profile and are of commercial value. Extraction of high purity steviol glycosides is expensive and the yields are low.

Thus, there is a need in the art for methods for obtaining steviol glycosides, and particularly for methods, especially-low cost methods, for obtaining steviol glycosides that result in increased yields of steviol glycosides.

The enzymatic conversion of steviol glycosides such as Rebaudioside A and stevioside into other steviol glycosides, most notably Rebaudioside M, is known in the art, however these conversions typically utilize a high purity starting material with the aim of investigating a specific conversion (see for example US 2020/0032227 A1, US 2020/0157594 A1, WO 2022/115527 A1, WO 2023/121426 A1 and WO 2018/144679 A3).

### SUMMARY OF THE DISCLOSURE

Provided herein are methods for obtaining steviol glycosides from stevia material using an enzyme mixture, wherein the stevia material can be an extract, co-product, or waste product from a stevia extraction process. The present invention has been based in part on the discoveries that the enzymatic conversion can be made to operate efficiently on low-grade and/or complex mixtures of starting materials, that the enzymatic conversion can be implemented to increase the efficiency of the steviol glycoside extraction process, and that the enzymes may be used to perform desirable and hitherto unidentified transformations of minor steviol glycosides that may be present within raw stevia extracts.

According to a first aspect of the disclosure, there is provided a method for obtaining one or more steviol glycosides, the method comprising the steps of adding an enzyme mixture to a stevia material; incubating the enzyme mixture and stevia material; and obtaining the one or more steviol glycosides from the enzyme mixture and stevia material.

In one embodiment of the first aspect of the disclosure, the stevia material has a total steviol glycoside content of ≤90% on a dry weight basis.

In another embodiment of the first aspect of the disclosure, the stevia material comprises rebaudioside A, stevioside and rebaudioside D.

In yet another embodiment of the first aspect of the disclosure, the stevia material comprises rubusoside and/or a rhamnosyl-containing steviol glycoside and/or a xylosyl-containing steviol glycoside.

Typically, the method is performed as part of an extraction process. Typically, the stevia material is an extract, co-product, or waste product. Thus, there is provided a method for obtaining one or more steviol glycosides, the method comprising the steps of adding an enzyme mixture to a stevia material during an extraction process, wherein the stevia material is an extract, co-product, or waste product; incubating the enzyme mixture and stevia material; and obtaining one or more steviol glycoside from the enzyme mixture and stevia material.

In one embodiment, a mixture of one or more steviol glycosides is obtained. Thus, there is provided a method for obtaining a mixture of one or more steviol glycosides, the method comprising the steps of adding an enzyme mixture to a stevia material during an extraction process, wherein the stevia material is an extract, co-product, or waste product; incubating the enzyme mixture and stevia material; and obtaining a mixture of one or more steviol glycosides from the enzyme mixture and stevia material.

In one embodiment, a purified mixture of one or more steviol glycosides is obtained. Thus, there is provided a method for obtaining a purified mixture of one or more steviol glycosides, the method comprising the steps of adding an enzyme mixture to a stevia material during an extraction process, wherein the stevia material is an extract, co-product, or waste product; incubating the enzyme mixture and stevia material; obtaining a mixture of one or more steviol glycosides from the enzyme mixture and stevia material; and purifying the mixture of the one or more steviol glycosides.

According to a second aspect of the disclosure, there is provided a product comprising one or more steviol glycosides, wherein the product is obtained or obtainable by a method of the first aspect of the disclosure. Thus, there is provided a product comprising one or more steviol glycosides produced by adding an enzyme mixture to a stevia material during an extraction process, wherein the stevia material is an extract, co-product, or waste product; incubating the enzyme mixture and stevia material; and obtaining one or more steviol glycosides from the enzyme mixture and stevia material.

These and other features, objects, and advantages of the present disclosure will become better understood from the description that follows. In the description, reference is made to the accompanying drawings, which form a part hereof and in which there is shown by way of illustration, not limitation, embodiments of the disclosure. The description of preferred embodiments is not intended to limit the disclosure to cover all modifications, equivalents, and alternatives. Reference should therefore be made to the claims recited herein for interpreting the scope of the disclosure.

### BRIEF DESCRIPTION OF THE DRAWINGS

The disclosure will be better understood, and features, aspects, and advantages other than those set forth above, will become apparent when consideration is given to the following detailed description thereof. Such detailed description refers to the following drawings.

FIG. 1 shows a diagram of the conversion of Rebaudioside C to Rebaudioside K or Rebaudioside N2.

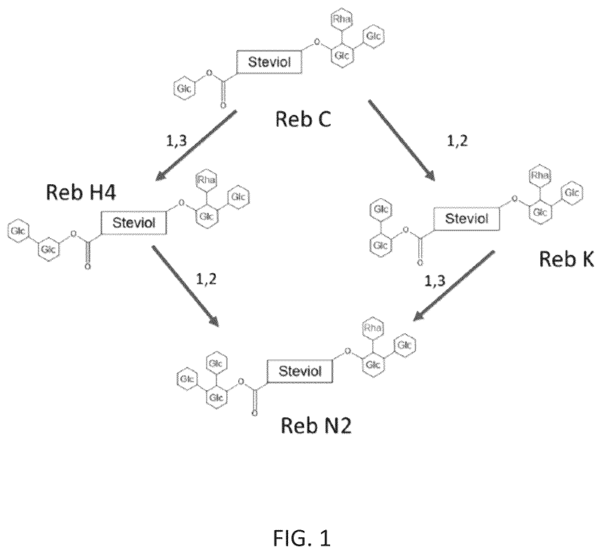

FIG. 2 shows an LC-MS output of Rebaudioside H4 production from Rebaudioside C with sucrose synthase and 1-3 beta-glycosylation enzyme. Creation of product eluted between 9-10 minutes.

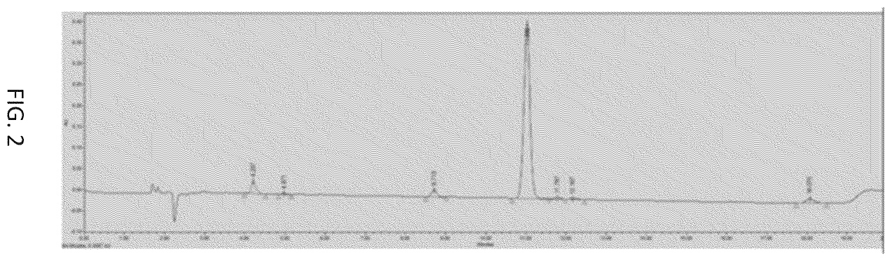

FIG. 3 shows an LC-MS output of Rebaudioside K production from Rebaudioside C with sucrose synthase and 1-2 beta-glycosylation enzyme. Creation of product eluting between 4-5 minutes.

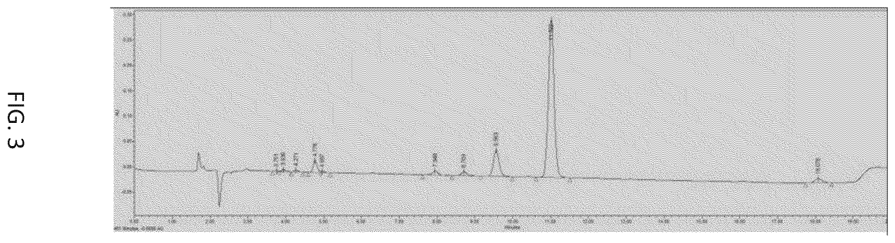

FIG. 4 shows an LC-MS output of the creation of Rebaudioside N2 from Rebaudioside C treatment with sucrose synthase, ADP, sucrose, and 1-2 and 1-3 beta-glycosylation enzymes. Rebaudioside N2 is mainly generated through Rebaudioside K as shown by chromatograms at time points 1, 3, and 24 hours of reaction. Rebaudioside N2 eluted at approximately 4 minutes, Rebaudioside K initially eluted between 4-5 minutes, and the consumption of Rebaudioside K and Rebaudioside C to produce highly pure Rebaudioside N2 eluted at 24 hours. A small peak eluting at approximately 4.8 minutes in the 24 hour reaction product is Rebaudioside M and Stevioside produced by the glycosylation of residual Rebaudioside A and D in the starting material.

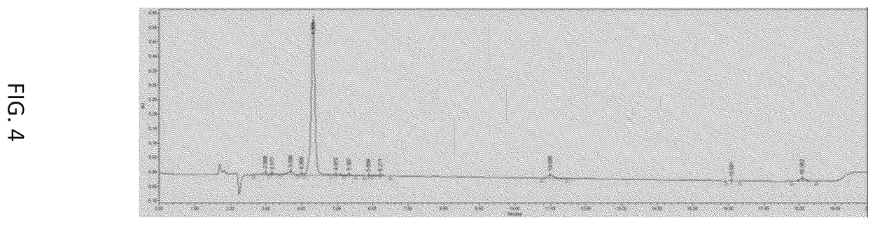

FIG. 5A-5C shows an LC-MS output of a stevia extract treated with ADP (FIG. 5A), sucrose synthase (FIG. 5B), and sucrose and one or both of 1-2 and 1-3 glycosyltransferases (FIG. 5C).

FIG. 6 shows starting material with Rebaudioside F elution at 22.086 minutes.

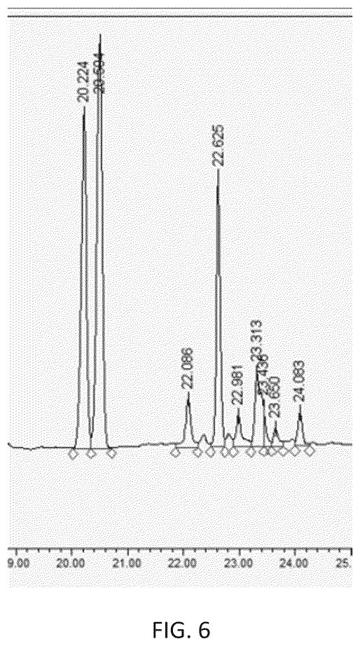

FIG. 7 shows an LC-MS output of a reacted Rebaudioside F generating a peak at 16.785 minutes, which represents a glycoside with 1 xylosyl and 5 glycosyl linkages. Unmarked peaks at 17.3 and 18.5 minutes represent minor isomers of this product, and an unmarked peak at 15.4 minutes represents a product with 1 xylosyl and 4 glucosyl residues.

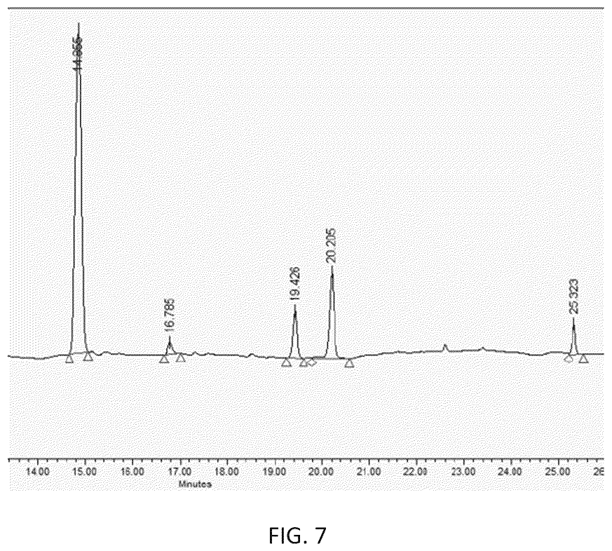

### DETAILED DESCRIPTION

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### DEFINITIONS

As used in the specification, articles “a” and “an” refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. Typically, the term “about” in association with a numerical value means that the numerical value can vary by plus or minus 5% or less of the numerical value.

Throughout this specification, unless the context requires otherwise, the word “comprise” and “include” and variations thereof (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are considered to be expressly stated in this disclosure.

Unless otherwise defined, all technical terms used herein have the same meaning as commonly understood by a person of ordinary skill in the art to which this disclosure belongs.

The term “stevia,” as used herein, refers to a natural sweetener containing steviol glycosides. The term “stevia plant,” as used herein, refers to the plant species Stevia rebaudiana. Stevia plants contain steviol glycosides, which are extracted for their sweet taste.

The term “steviol glycoside,” as used herein, refers to a naturally occurring sweet compound derived from a stevia plant. Steviol glycosides include, but are not limited to Stevioside, Rebaudiosides, and Rubusoside. Stevioside (STV) is a compound mainly responsible for the sweet taste in stevia plants. Rebaudiosides are steviol glycosides derived from stevia plants. Rebaudiosides include, but are not limited to, Rebaudioside A, Rebaudioside B, Rebaudioside C, Rebaudioside D, Rebaudioside E, Rebaudioside F, Rebaudioside I, and Rebaudioside M. Rebaudiosides are frequently referred to as Reb (e.g., Reb A, Reb B, or Reb C). Rebaudioside A is the sweetest, and second most prevalent steviol glycoside in stevia plants. Rubusoside (RU) is a naturally occurring sweet steviol glycoside derived from stevia plant in small quantities. Rubusoside is often derived from Rubus suavissimus and is used as an alternative to sugar.

The methods disclosed herein were developed using enzymes that are capable of converting stevioside to Rebaudioside M. The methods can be used with steviol glycosides that contain sugars other than only glucose, for example, Rebaudioside K could be obtained from Rebaudioside C or Rebaudioside N from Rebaudioside J. Additionally, mass can be added to other glucosides.

The term “enzyme mixture” as used herein, refers to any mixture comprising one or more glycosyltransferase enzymes. Typically, the “enzyme mixture” includes glycosyltransferase enzymes and sucrose synthase enzymes, as disclosed in U.S. Patent Application Publication No. US20200032227 A1.

The term “stevia material,” as used herein, refers to any material comprising one or more steviol glycosides. Typically, the stevia material is an extract, co-product, or waste product obtained from a stevia extraction process. A stevia extract or extract is a product of the stevia plant extraction process containing 40-95% steviol glycosides by dry weight. The stevia extract or extract can be a product that has not been purified. A stevia waste product or waste product is an unpurified product generated from the first step in the stevia extraction product. Enrichment of a steviol glycoside cannot be obtained by crystallization of a stevia waste product. The term “mother liquor,” as used herein, refers to a co-product stream obtained from crystallization of a stevia stream. Mother liquors contain steviol glycosides in the amount of 40-95% by dry weight.

The term “minimally processed stevia extract,” as used herein, is understood to include the extraction from stevia leaves with water and/or alcohol, flocculation with metal salts, such as iron sulfate and calcium hydroxide, adsorption of an aqueous solution to a macroporous resin and subsequent desorption using alcohol, ion-exchange resins, activated carbon, or filtration, including diafiltration. Minimally processed stevia extracts contain steviol glycosides in the amount of 50-95% of dry weight.

The term “mass gain efficiency,” as used herein, refers to an increase to percent of steviol glycoside, on a dry weight basis, in the stevia material.

### Methods of Enzymatic Conversion

As stated above, a first aspect of the disclosure provides a method for obtaining one or more steviol glycosides, the method comprising the steps of (a) adding an enzyme mixture to a stevia material; (b) incubating the enzyme mixture and stevia material; and (c) obtaining the one or more steviol glycosides from the enzyme mixture and stevia material.

Typically, the stevia material is an extract, co-product, or waste product obtained from a stevia extraction process.

As noted above, the present disclosure has been based in part on the discovery that the enzymatic conversion can be made to operate efficiently on low-grade starting materials.

Accordingly, in a first mode of the first aspect of the disclosure, the stevia material has a total steviol glycoside content of ≤90% on a dry weight basis. In one embodiment of the first mode, the stevia material has a total steviol glycoside content of ≤85% or ≤80% on a dry weight basis. Typically, the stevia material used in the first mode has a total steviol glycoside content of ≥40% on a dry weight basis. More typically, the stevia material has a total steviol glycoside content of ≥50% or ≥60% on a dry weight basis. Thus, it may be envisaged for example that the stevia material has a total steviol glycoside content of from 40% to 90%, or from 50% to 85%, or from 60% to 85%, or from 60% to 80%, all on a dry weight basis.

Typically, the stevia material used in the first mode comprises a mixture of steviol glycosides. For example, the stevia material used in the first mode may comprise rebaudioside A, stevioside, optionally one or more additional steviol glycosides selected from the group consisting of rebaudioside D, rebaudioside E, rebaudioside I, rebaudioside M and rubusoside, and optionally one or more further steviol glycosides.

Typically, the total content of rebaudioside A and stevioside in the stevia material used in the first mode is from 40 to 100 wt % of the total steviol glycoside content of the stevia material. More typically, the total content of rebaudioside A and stevioside is from 50 to 95 wt % of the total steviol glycoside content of the stevia material. More typically still, the total content of rebaudioside A and stevioside is from 60 to 90 wt %.

As will be appreciated, where the stevia material used in the first mode comprises rebaudioside A and stevioside, the rebaudioside A and stevioside may be present in any ratio, e.g. from 1:99 to 99:1 on a weight basis.

Typically, where the total content of rebaudioside A and stevioside in the stevia material used in the first mode is from 40 to 100 wt % of the total steviol glycoside content of the stevia material, the rebaudioside A content is from 5 to 95 wt % of the total steviol glycoside content and the stevioside content is from 5 to 95 wt %. More typically in such an embodiment, the rebaudioside A content is from 10 to 85 wt % and the stevioside content is from 10 to 85 wt %. Yet more typically, the rebaudioside A content is from 20 to 80 wt % and the stevioside content is from 10 to 60 wt %. More typically still, the rebaudioside A content is from 35 to 60 wt % and the stevioside content is from 15 to 50 wt %.

Where the stevia material used in the first mode comprises rebaudioside D, typically the content of rebaudioside D in the stevia material is from 0.1 to 50 wt % of the total steviol glycoside content of the stevia material. More typically, the rebaudioside D content is from 0.5 to 15 wt % of the total steviol glycoside content of the stevia material. Yet more typically, the rebaudioside D content is from 1 to 10 wt % of the total steviol glycoside content of the stevia material. More typically still, the rebaudioside D content is from 2 to 5 wt % of the total steviol glycoside content of the stevia material.

Moreover, as noted above, the present disclosure has further been based in part on the discovery that the enzymatic conversion can be made to operate efficiently on complex mixtures of starting materials. Accordingly, in a second mode of the first aspect of the disclosure, the stevia material comprises rebaudioside A, stevioside and rebaudioside D. In one embodiment of the second mode of the first aspect of the disclosure, the stevia material further comprises one or more additional steviol glycosides selected from the group consisting of rebaudioside E, rebaudioside I, rebaudioside M and rubusoside, and optionally one or more further steviol glycosides. For example, in one embodiment, the stevia material comprises at least rebaudioside A, stevioside, rebaudioside D and rebaudioside M. In another embodiment, the stevia material comprises at least rebaudioside A, stevioside, rebaudioside D and rubusoside.

As will be appreciated, the stevia material used in the second mode may comprise rebaudioside A, stevioside and rebaudioside D in any ratio.

Typically, the total content of rebaudioside A and stevioside in the stevia material used in the second mode of the first aspect of the disclosure is from 40 to 99.9 wt % of the total steviol glycoside content of the stevia material. More typically, the total content of rebaudioside A and stevioside is from 50 to 95 wt % of the total steviol glycoside content of the stevia material. More typically still, the total content of rebaudioside A and stevioside is from 60 to 90 wt %.

Typically, where the total content of rebaudioside A and stevioside in the stevia material used in the second mode is from 40 to 99.9 wt % of the total steviol glycoside content of the stevia material, the rebaudioside A content is from 5 to 95 wt % of the total steviol glycoside content and the stevioside content is from 5 to 95 wt %. More typically in such an embodiment, the rebaudioside A content is from 10 to 85 wt % and the stevioside content is from 10 to 85 wt %. Yet more typically, the rebaudioside A content is from 20 to 80 wt % and the stevioside content is from 10 to 60 wt %. More typically still, the rebaudioside A content is from 35 to 60 wt % and the stevioside content is from 15 to 50 wt %.

Typically the rebaudioside D content of the stevia material used in the second mode of the first aspect of the disclosure is from 0.1 to 50 wt % of the total steviol glycoside content of the stevia material. More typically, the rebaudioside D content is from 0.5 to 15 wt % of the total steviol glycoside content of the stevia material. Yet more typically, the rebaudioside D content is from 1 to 10 wt % of the total steviol glycoside content of the stevia material. More typically still, the rebaudioside D content is from 2 to 5 wt % of the total steviol glycoside content of the stevia material.

Where the stevia material used in the first or the second mode of the disclosure comprises rebaudioside E, typically, the rebaudioside E content is from 0.01 to 10 wt % of the total steviol glycoside content of the stevia material. More typically, the rebaudioside E content is from 0.1 to 5 wt % of the total steviol glycoside content of the stevia material. More typically still, the rebaudioside E content is from 0.5 to 1.5 wt % of the total steviol glycoside content of the stevia material.

Where the stevia material used in the first or the second mode of the disclosure comprises rebaudioside I, typically, the rebaudioside I content is from 0.01 to 10 wt % of the total steviol glycoside content of the stevia material. More typically, the rebaudioside I content is from 0.05 to 2 wt % of the total steviol glycoside content of the stevia material. More typically still, the rebaudioside I content is from 0.1 to 0.5 wt % of the total steviol glycoside content of the stevia material.

Where the stevia material used in the first or the second mode of the disclosure comprises rebaudioside M, typically the rebaudioside M content is from 0.01 to 20 wt % of the total steviol glycoside content of the stevia material. More typically, the rebaudioside M content is from 0.1 to 15 wt % of the total steviol glycoside content of the stevia material. More typically still, the rebaudioside M content is from 0.2 to 10 wt % of the total steviol glycoside content of the stevia material.

Where the stevia material used in the first or the second mode of the disclosure comprises rubusoside, typically, the rubusoside content is from 0.01 to 10 wt % of the total steviol glycoside content of the stevia material. More typically, the rubusoside content is from 0.1 to 5 wt % of the total steviol glycoside content of the stevia material. More typically still, the rubusoside content is from 0.2 to 2 wt % of the total steviol glycoside content of the stevia material.

Typically in accordance with the second mode of the first aspect of the disclosure, the stevia material has a total steviol glycoside content of ≤95% on a dry weight basis. Typically, the stevia material used in the second mode has a total steviol glycoside content of ≥40% on a dry weight basis. Thus, for instance, the stevia material may have a total steviol glycoside content of from 40% to 95% on a dry weight basis.

In one embodiment, the method is in accordance with both the first mode and the second mode of the first aspect of the disclosure. In other words, there is provided a method for obtaining one or more steviol glycosides, the method comprising the steps of adding an enzyme mixture to a stevia material; incubating the enzyme mixture and stevia material; and obtaining the one or more steviol glycosides from the enzyme mixture and stevia material, wherein the stevia material comprises rebaudioside A, stevioside and rebaudioside D, and wherein the stevia material has a total steviol glycoside content of ≤90% on a dry weight basis.

In addition, as discussed above, it has been discovered that enzymes may be used to perform desirable and hitherto unidentified transformations of minor steviol glycosides that may be present within raw stevia extracts. For example. Using enzymes developed to convert stevioside to Rebaudioside M, it was demonstrated that these enzymes would readily glycosylate steviol glycosides at the glucose directly linked to the steviol core selecting for beta 1-2 and beta 1-3 linkages. For instance, rubusoside contains two glucosyl residues attached to the C-13 and C-19 positions of the steviol core that can be fully glycosylated to Rebaudioside M with these enzymes gaining a total of four glucosyl groups. Despite an unoptimized process, when an enzyme mixture as described herein is applied to a minimally processed stevia extract, the recovered mass of steviol glycosides is expected to rise from about 80% to about 90%, due to mass gain efficiency. When applied to mother liquor from the extraction process, for every 100 kg of steviol glycoside in the mother liquor, 104 kg of steviol glycoside can be recovered after reaction.

Furthermore, steviol glycosides may have saccharides other than glucose attached, such as rhamnosyl- or xylosyl-containing carbohydrates. These were first discovered in Rebaudioside C and Rebaudioside F, respectively. With the implementation of the disclosed methods, an enzyme mixture as described herein can add beta 1-2 and beta 1-3 glucosyl-linkages to rhamosyl- and xylosyl-containing steviol glycosides as well. Based on liquid chromatography-mass spectrometry data it can be determined that at least three steviol glycosides with 5 glucosyl and 1 xylosyl or rhamnosyl residue are created by the methods disclosed herein. The methods, coupled with the enzymes disclosed in U.S. Patent Application Publication No. US20200032227 A1, can result in full glycosylation of at least 2 isomers of Rebaudiosides C and F.

Applying these methods to minimally processed stevia extract or mother liquor, various steviol glycosides such as Rebaudioside C, Rebaudioside F, and rubusoside are glycosylated multiple times by enzymes initially designed for stevioside-to-Rebaudioside M conversion. Bioconversion was carried out at optimal enzyme, sucrose, and ADP concentrations to achieve the mass gain efficiency. Glucose was added to steviol glycosides with rhamnosyl and xylosyl residues as shown in Example 5. This resulted in increases in yield of steviol glycosides from a stevia processing facility.

Accordingly, in a third mode of the first aspect of the disclosure, the stevia material comprises rubusoside and/or a rhamnosyl-containing steviol glycoside and/or a xylosyl-containing steviol glycoside.

In one embodiment of the third mode, the stevia material comprises rubusoside. The rubusoside may have been extracted from stevia plant or alternately may have been extracted from Rubus suavissimus.

In one embodiment, the rubusoside is a major component of the stevia material. For example, the stevia material may contain ≥50%, ≥75%, ≥85% or ≥95% rubusoside on a dry weight basis.

Alternately, the rubusoside may be a minor component of the stevia material. For example, the rubusoside content of the stevia material may be ≤10 wt %, ≤5 wt % or ≤2 wt % of the total steviol glycoside content of the stevia material. Typically, where the rubusoside is a minor component of the stevia material, the rubusoside content is ≥0.01 wt %, ≥0.1 wt % or ≥0.5 wt % of the total steviol glycoside content of the stevia material. Thus, it may be envisaged for example that the stevia material has a rubusoside content of from 0.01 wt % to 10 wt %, or from 0.1 wt % to 5 wt %, or from 0.5 wt % to 2 wt % of the total steviol glycoside content of the stevia material.

In one embodiment, where the rubusoside is a minor component of the stevia material, the stevia material has a total steviol glycoside content of ≤95% on a dry weight basis. Typically, where the rubusoside is a minor component of the stevia material, the stevia material has a total steviol glycoside content of ≥40% on a dry weight basis. Thus, for instance, the stevia material may have a total steviol glycoside content of from 40% to 95% on a dry weight basis. Optionally, where the rubusoside is a minor component of the stevia material, the method is also in accordance with the first mode of the first aspect of the disclosure. In other words, the stevia material has a total steviol glycoside content of ≤90% on a dry weight basis.

Typically, where the rubusoside is a minor component of the stevia material, the stevia material further comprises rebaudioside A, stevioside, optionally one or more additional steviol glycosides selected from the group consisting of rebaudioside D, rebaudioside E, rebaudioside I, and rebaudioside M, and optionally one or more further steviol glycosides.

Typically, where the rubusoside is a minor component of the stevia material, the total content of rebaudioside A and stevioside in the stevia material used is from 40 to 99.9 wt % of the total steviol glycoside content of the stevia material. More typically, the total content of rebaudioside A and stevioside is from 50 to 95 wt % of the total steviol glycoside content of the stevia material. More typically still, the total content of rebaudioside A and stevioside is from 60 to 90 wt %. As will be appreciated, where the stevia material used comprises rebaudioside A and stevioside, the rebaudioside A and stevioside may be present in any ratio, e.g. from 1:99 to 99:1 on a weight basis.

Typically, where the rubusoside is a minor component of the stevia material, and where the total content of rebaudioside A and stevioside in the stevia material used is from 40 to 99.9 wt % of the total steviol glycoside content of the stevia material, the rebaudioside A content is from 5 to 95 wt % of the total steviol glycoside content and the stevioside content is from 5 to 95 wt %. More typically in such an embodiment, the rebaudioside A content is from 10 to 85 wt % and the stevioside content is from 10 to 85 wt %. Yet more typically, the rebaudioside A content is from 20 to 80 wt % and the stevioside content is from 10 to 60 wt %. More typically still, the rebaudioside A content is from 35 to 60 wt % and the stevioside content is from 15 to 50 wt %.

Where the rubusoside is a minor component of the stevia material, and where the stevia material used comprises rebaudioside D, typically the content of rebaudioside D in the stevia material is from 0.1 to 50 wt % of the total steviol glycoside content of the stevia material. More typically, the rebaudioside D content is from 0.5 to 15 wt % of the total steviol glycoside content of the stevia material. Yet more typically, the rebaudioside D content is from 1 to 10 wt % of the total steviol glycoside content of the stevia material. More typically still, the rebaudioside D content is from 2 to 5 wt % of the total steviol glycoside content of the stevia material.

Where the rubusoside is a minor component of the stevia material, and where the stevia material used comprises rebaudioside E, typically, the rebaudioside E content is from 0.01 to 10 wt % of the total steviol glycoside content of the stevia material. More typically, the rebaudioside E content is from 0.1 to 5 wt % of the total steviol glycoside content of the stevia material. More typically still, the rebaudioside E content is from 0.5 to 1.5 wt % of the total steviol glycoside content of the stevia material.

Where the rubusoside is a minor component of the stevia material, and where the stevia material used comprises rebaudioside I, typically, the rebaudioside I content is from 0.01 to 10 wt % of the total steviol glycoside content of the stevia material. More typically, the rebaudioside I content is from 0.05 to 2 wt % of the total steviol glycoside content of the stevia material. More typically still, the rebaudioside I content is from 0.1 to 0.5 wt % of the total steviol glycoside content of the stevia material.

Where the rubusoside is a minor component of the stevia material, and where the stevia material used comprises rebaudioside M, typically the rebaudioside M content is from 0.01 to 20 wt % of the total steviol glycoside content of the stevia material. More typically, the rebaudioside M content is from 0.1 to 15 wt % of the total steviol glycoside content of the stevia material. More typically still, the rebaudioside M content is from 0.2 to 10 wt % of the total steviol glycoside content of the stevia material.

In one embodiment, where the rubusoside is a minor component of the stevia material, the method is also in accordance with the second mode of the first aspect of the disclosure. In other words, the stevia material comprises rebaudioside A, stevioside, rebaudioside D and, as a minor component, rubusoside.

In another embodiment of the third mode, the stevia material comprises a rhamnosyl-containing steviol glycoside such as rebaudioside C, dulcoside A and/or dulcoside B. Typically in such an embodiment, the stevia material comprises rebaudioside C.

In one embodiment, the rhamnosyl-containing steviol glycoside is a major component of the stevia material. For example, the stevia material may contain ≥50%, ≥75%, ≥85% or ≥95% of the rhamnosyl-containing steviol glycoside on a dry weight basis. For instance, the stevia material may contain ≥75%, ≥85% or ≥95% of rebaudioside C on a dry weight basis.

Alternately, the rhamnosyl-containing steviol glycoside may be a minor component of the stevia material. Typically in such embodiments, the rhamnosyl-containing steviol glycoside content (e.g. the rebaudioside C content) of the stevia material is <50 wt % of the total steviol glycoside content of the stevia material. For example, the rhamnosyl-containing steviol glycoside content (e.g. the rebaudioside C content) of the stevia material may be ≤25 wt %, ≤10 wt % or ≤5 wt % of the total steviol glycoside content of the stevia material. Typically, where the rhamnosyl-containing steviol glycoside is a minor component of the stevia material, the rhamnosyl-containing steviol glycoside content (e.g. the rebaudioside C content) is ≥0.1 wt %, ≥0.5 wt % or ≥1 wt % of the total steviol glycoside content of the stevia material. Thus, it may be envisaged for example that the stevia material has a rhamnosyl-containing steviol glycoside content (e.g. a rebaudioside C content) of from 0.1 wt % to 25 wt %, or from 0.5 wt % to 10 wt %, or from 1 wt % to 5 wt % of the total steviol glycoside content of the stevia material.

In one embodiment, where the rhamnosyl-containing steviol glycoside is a minor component of the stevia material, the stevia material has a total steviol glycoside content of ≤95% on a dry weight basis. Typically, where the rhamnosyl-containing steviol glycoside is a minor component of the stevia material, the stevia material has a total steviol glycoside content of ≥40% on a dry weight basis. Thus, for instance, the stevia material may have a total steviol glycoside content of from 40% to 95% on a dry weight basis. Optionally, where the rhamnosyl-containing steviol glycoside is a minor component of the stevia material, the method is also in accordance with the first mode of the first aspect of the disclosure. In other words, the stevia material has a total steviol glycoside content of ≤90% on a dry weight basis.

Typically, where the rhamnosyl-containing steviol glycoside is a minor component of the stevia material, the stevia material further comprises rebaudioside A, stevioside, optionally one or more additional steviol glycosides selected from the group consisting of rebaudioside D, rebaudioside E, rebaudioside I, rebaudioside M and rubusoside, and optionally one or more further steviol glycosides.

Typically, where the rhamnosyl-containing steviol glycoside is a minor component of the stevia material, the total content of rebaudioside A and stevioside in the stevia material used is from 40 to 99.9 wt % of the total steviol glycoside content of the stevia material. More typically, the total content of rebaudioside A and stevioside is from 50 to 95 wt % of the total steviol glycoside content of the stevia material. More typically still, the total content of rebaudioside A and stevioside is from 60 to 90 wt %. As will be appreciated, where the stevia material used comprises rebaudioside A and stevioside, the rebaudioside A and stevioside may be present in any ratio, e.g. from 1:99 to 99:1 on a weight basis.

Typically, where the rhamnosyl-containing steviol glycoside is a minor component of the stevia material, and where the total content of rebaudioside A and stevioside in the stevia material used is from 40 to 99.9 wt % of the total steviol glycoside content of the stevia material, the rebaudioside A content is from 5 to 95 wt % of the total steviol glycoside content and the stevioside content is from 5 to 95 wt %. More typically in such an embodiment, the rebaudioside A content is from 10 to 85 wt % and the stevioside content is from 10 to 85 wt %. Yet more typically, the rebaudioside A content is from 20 to 80 wt % and the stevioside content is from 10 to 60 wt %. More typically still, the rebaudioside A content is from 35 to 60 wt % and the stevioside content is from 15 to 50 wt %.

Where the rhamnosyl-containing steviol glycoside is a minor component of the stevia material, and where the stevia material used comprises rebaudioside D, typically the content of rebaudioside D in the stevia material is from 0.1 to 50 wt % of the total steviol glycoside content of the stevia material. More typically, the rebaudioside D content is from 0.5 to 15 wt % of the total steviol glycoside content of the stevia material. Yet more typically, the rebaudioside D content is from 1 to 10 wt % of the total steviol glycoside content of the stevia material. More typically still, the rebaudioside D content is from 2 to 5 wt % of the total steviol glycoside content of the stevia material.

Where the rhamnosyl-containing steviol glycoside is a minor component of the stevia material, and where the stevia material used comprises rebaudioside E, typically, the rebaudioside E content is from 0.01 to 10 wt % of the total steviol glycoside content of the stevia material. More typically, the rebaudioside E content is from 0.1 to 5 wt % of the total steviol glycoside content of the stevia material. More typically still, the rebaudioside E content is from 0.5 to 1.5 wt % of the total steviol glycoside content of the stevia material.

Where the rhamnosyl-containing steviol glycoside is a minor component of the stevia material, and where the stevia material used comprises rebaudioside I, typically, the rebaudioside I content is from 0.01 to 10 wt % of the total steviol glycoside content of the stevia material. More typically, the rebaudioside I content is from 0.05 to 2 wt % of the total steviol glycoside content of the stevia material. More typically still, the rebaudioside I content is from 0.1 to 0.5 wt % of the total steviol glycoside content of the stevia material.

Where the rhamnosyl-containing steviol glycoside is a minor component of the stevia material, and where the stevia material used comprises rebaudioside M, typically the rebaudioside M content is from 0.01 to 20 wt % of the total steviol glycoside content of the stevia material. More typically, the rebaudioside M content is from 0.1 to 15 wt % of the total steviol glycoside content of the stevia material. More typically still, the rebaudioside M content is from 0.2 to 10 wt % of the total steviol glycoside content of the stevia material.

Where the rhamnosyl-containing steviol glycoside is a minor component of the stevia material, and where the stevia material used comprises rubusoside, typically, the rubusoside content is from 0.01 to 10 wt % of the total steviol glycoside content of the stevia material. More typically, the rubusoside content is from 0.1 to 5 wt % of the total steviol glycoside content of the stevia material. More typically still, the rubusoside content is from 0.2 to 2 wt % of the total steviol glycoside content of the stevia material.

In one embodiment, where the rhamnosyl-containing steviol glycoside is a minor component of the stevia material, the method is also in accordance with the second mode of the first aspect of the disclosure. In other words, the stevia material comprises rebaudioside A, stevioside, rebaudioside D and, as a minor component, a rhamnosyl-containing steviol glycoside such as rebaudioside C.

In yet another embodiment of the third mode, the stevia material comprises a xylosyl—containing steviol glycoside such as rebaudioside F.

In one embodiment, the xylosyl-containing steviol glycoside is a major component of the stevia material. For example, the stevia material may contain ≥50%, ≥75%, ≥85% or ≥95% of the xylosyl-containing steviol glycoside on a dry weight basis. For instance, the stevia material may contain ≥75%, ≥85% or ≥95% of rebaudioside F on a dry weight basis.

Alternately, the xylosyl-containing steviol glycoside may be a minor component of the stevia material. Typically in such embodiments, the xylosy-containing steviol glycoside content (e.g. the rebaudioside C content) of the stevia material is <50 wt % of the total steviol glycoside content of the stevia material. For example, the xylosyl-containing steviol glycoside content (e.g. the rebaudioside F content) of the stevia material may be ≤15 wt %, ≤10 wt % or ≤5 wt % of the total steviol glycoside content of the stevia material. Typically, where the xylosyl-containing steviol glycoside is a minor component of the stevia material, the xylosyl-containing steviol glycoside content (e.g. the rebaudioside F content) is ≥0.1 wt %, ≥0.5 wt % or ≥1 wt % of the total steviol glycoside content of the stevia material. Thus, it may be envisaged for example that the stevia material has a xylosyl-containing steviol glycoside content (e.g. a rebaudioside F content) of from 0.1 wt % to 15 wt %, or from 0.5 wt % to 10 wt %, or from 1 wt % to 5 wt % of the total steviol glycoside content of the stevia material.

In one embodiment, where the xylosyl-containing steviol glycoside is a minor component of the stevia material, the stevia material has a total steviol glycoside content of ≤95% on a dry weight basis. Typically, where the xylosyl-containing steviol glycoside is a minor component of the stevia material, the stevia material has a total steviol glycoside content of ≥40% on a dry weight basis. Thus, for instance, the stevia material may have a total steviol glycoside content of from 40% to 95% on a dry weight basis. Optionally, where the xylosyl-containing steviol glycoside is a minor component of the stevia material, the method is also in accordance with the first mode of the first aspect of the disclosure. In other words, the stevia material has a total steviol glycoside content of ≤90% on a dry weight basis.

Typically, where the xylosyl-containing steviol glycoside is a minor component of the stevia material, the stevia material further comprises rebaudioside A, stevioside, optionally one or more additional steviol glycosides selected from the group consisting of rebaudioside D, rebaudioside E, rebaudioside I, rebaudioside M and rubusoside, and optionally one or more further steviol glycosides.

Typically, where the xylosyl-containing steviol glycoside is a minor component of the stevia material, the total content of rebaudioside A and stevioside in the stevia material used is from 40 to 99.9 wt % of the total steviol glycoside content of the stevia material. More typically, the total content of rebaudioside A and stevioside is from 50 to 95 wt % of the total steviol glycoside content of the stevia material. More typically still, the total content of rebaudioside A and stevioside is from 60 to 90 wt %. As will be appreciated, where the stevia material used comprises rebaudioside A and stevioside, the rebaudioside A and stevioside may be present in any ratio, e.g. from 1:99 to 99:1 on a weight basis.

Typically, where the xylosyl-containing steviol glycoside is a minor component of the stevia material, and where the total content of rebaudioside A and stevioside in the stevia material used is from 40 to 99.9 wt % of the total steviol glycoside content of the stevia material, the rebaudioside A content is from 5 to 95 wt % of the total steviol glycoside content and the stevioside content is from 5 to 95 wt %. More typically in such an embodiment, the rebaudioside A content is from 10 to 85 wt % and the stevioside content is from 10 to 85 wt %. Yet more typically, the rebaudioside A content is from 20 to 80 wt % and the stevioside content is from 10 to 60 wt %. More typically still, the rebaudioside A content is from 35 to 60 wt % and the stevioside content is from 15 to 50 wt %.

Where the xylosyl-containing steviol glycoside is a minor component of the stevia material, and where the stevia material used comprises rebaudioside D, typically the content of rebaudioside D in the stevia material is from 0.1 to 50 wt % of the total steviol glycoside content of the stevia material. More typically, the rebaudioside D content is from 0.5 to 15 wt % of the total steviol glycoside content of the stevia material. Yet more typically, the rebaudioside D content is from 1 to 10 wt % of the total steviol glycoside content of the stevia material. More typically still, the rebaudioside D content is from 2 to 5 wt % of the total steviol glycoside content of the stevia material.

Where the xylosyl-containing steviol glycoside is a minor component of the stevia material, and where the stevia material used comprises rebaudioside E, typically, the rebaudioside E content is from 0.01 to 10 wt % of the total steviol glycoside content of the stevia material. More typically, the rebaudioside E content is from 0.1 to 5 wt % of the total steviol glycoside content of the stevia material. More typically still, the rebaudioside E content is from 0.5 to 1.5 wt % of the total steviol glycoside content of the stevia material.

Where the xylosyl-containing steviol glycoside is a minor component of the stevia material, and where the stevia material used comprises rebaudioside I, typically, the rebaudioside I content is from 0.01 to 10 wt % of the total steviol glycoside content of the stevia material. More typically, the rebaudioside I content is from 0.05 to 2 wt % of the total steviol glycoside content of the stevia material. More typically still, the rebaudioside I content is from 0.1 to 0.5 wt % of the total steviol glycoside content of the stevia material.

Where the xylosyl-containing steviol glycoside is a minor component of the stevia material, and where the stevia material used comprises rebaudioside M, typically the rebaudioside M content is from 0.01 to 20 wt % of the total steviol glycoside content of the stevia material. More typically, the rebaudioside M content is from 0.1 to 15 wt % of the total steviol glycoside content of the stevia material. More typically still, the rebaudioside M content is from 0.2 to 10 wt % of the total steviol glycoside content of the stevia material.

Where the xylosyl-containing steviol glycoside is a minor component of the stevia material, and where the stevia material used comprises rubusoside, typically, the rubusoside content is from 0.01 to 10 wt % of the total steviol glycoside content of the stevia material. More typically, the rubusoside content is from 0.1 to 5 wt % of the total steviol glycoside content of the stevia material. More typically still, the rubusoside content is from 0.2 to 2 wt % of the total steviol glycoside content of the stevia material.

In one embodiment, where the xylosyl-containing steviol glycoside is a minor component of the stevia material, the method is also in accordance with the second mode of the first aspect of the disclosure. In other words, the stevia material comprises rebaudioside A, stevioside, rebaudioside D and, as a minor component, a xylosyl-containing steviol glycoside such as rebaudioside F.

In some embodiments, applicable to the first aspect and any of the modes as outlined above, the method for obtaining one or more steviol glycosides comprises adding an enzyme mixture to a stevia material during an extraction process, wherein the stevia material is an extract, co-product, or waste product; incubating the enzyme mixture and stevia material; and obtaining steviol glycoside from the enzyme mixture and stevia material.

In some embodiments, the stevia material comprises an extract, such as a minimally processed stevia extract. In some embodiments, minimally processed stevia extract contains 60 to 95% total steviol glycosides on a dry weight basis.

In some embodiments, the stevia material is a co-product product. In particular embodiments, the co-product product is a mother liquor.

In some embodiments, the mother liquor comprises about 40% to about 95% steviol glycoside on a dry weight basis.

Where the stevia material has a total steviol glycoside content of <100% on a dry weight basis, typically the remainder of the dry weight of the stevia material comprises one or more components selected from the group consisting of polysaccharides, carbohydrates, ash, polyphenols, flavonoids, proteins, amino acids, vitamins, fats and plant metabolites. For example, the remainder of the dry weight of the stevia material may comprise polysaccharides and/or carbohydrates, and at least one or more components selected from the group consisting of ash, polyphenols, flavonoids, proteins, amino acids, vitamins, fats and plant metabolites.

More typically, the remainder of the dry weight of the stevia material consists essentially of one or more other components selected from the group consisting of polysaccharides, carbohydrates, ash, polyphenols, flavonoids, proteins, amino acids, vitamins, fats and plant metabolites. For example, the remainder of the dry weight of the stevia material may consist essentially of polysaccharides and/or carbohydrates, and at least one or more components selected from the group consisting of ash, polyphenols, flavonoids, proteins, amino acids, vitamins, fats and plant metabolites.

As will be understood, the step of incubating the enzyme mixture and stevia material results in the glycosylation of one or more steviol glycosides within the stevia material. Typically, the step of incubating the enzyme mixture and stevia material results in the glucosylation of one or more steviol glycosides within the stevia material. More typically, the step of incubating the enzyme mixture and stevia material results in the glucosylation of one or more steviol glycosides within the stevia material via β1-2 and/or β1-3 linkages.

For example, where the stevia material comprises one or more of rubusoside, stevioside, rebaudioside A, rebaudioside D, rebaudioside E or rebaudioside I; rubusoside may be transformed into stevioside; stevioside may be transformed into rebaudioside A and/or rebaudioside E; rebaudioside A may be transformed into rebaudioside D and/or rebaudioside I; rebaudioside E may be transformed into rebaudioside D; rebaudioside D may be transformed into rebaudioside M; and rebaudioside I may be transformed into rebaudioside M. Moreover, chain reactions may occur, resulting for example in the formation of rebauioside M from rebaudioside A (via rebaudioside D and/or rebaudioside I), from rebaudioside E (via rebaudioside D), from stevioside (via rebaudiosides A, D, E and/or I), and/or from rubusoside (via stevioside and/or any of rebaudiosides A, D, E and/or I).

Similarly, where the stevia material comprises rebaudioside C, the rebaudioside C may be transformed into β1-2 and/or β1-3 glucosylated derivatives of rebaudioside C, such as rebaudioside K, rebaudioside N2 and/or rebaudioside H4. And where the stevia material comprises rebaudioside F, the rebaudioside F may be transformed into β1-2 and/or β1-3 glucosylated derivatives of rebaudioside F.

Typically, the one or more steviol glycosides obtained comprise a mixture of steviol glycosides. For example, the one or more steviol glycosides obtained may comprise a mixture of (i) one or more steviol glycosides that have been glycosylated by the incubation step and (ii) one or more residual steviol glycosides from the stevia material.

In some embodiments, the steviol glycosides obtained using the methods of the disclosure comprise Rebaudioside A, Rebaudioside D, Rebaudioside M, Rebaudioside E,

Rebaudioside I, Stevioside, and/or Rubusoside. Such embodiments are typical in accordance with the first or second mode of the disclosure, or the third mode where the stevia material comprises rubusoside, or the third mode where the rhamnosyl-containing steviol glycoside or the xylosyl-containing steviol glycoside is a minor component of the stevia material.

In further embodiments, the steviol glycosides obtained comprise Rebaudioside A,

Rebaudioside D, Rebaudioside I, and/or Rebaudioside M. Again, such embodiments are typical in accordance with the first or second mode of the disclosure, or the third mode where the stevia material comprises rubusoside, or the third mode where the rhamnosyl-containing steviol glycoside or the xylosyl-containing steviol glycoside is a minor component of the stevia material. In particular, such embodiments are typical where the stevia material comprises rebaudioside A and/or stevioside and/or rubusoside. In some embodiments, the steviol glycoside obtained comprises about 1% to about 38% Rebaudioside A on a dry weight basis. In one embodiment, the steviol glycoside obtained comprises about 11% Rebaudioside A. In some embodiments, the steviol glycoside obtained comprises about 10% to about 33% Rebaudioside D on a dry weight basis. In one embodiment, the steviol glycoside obtained comprises about 13% Rebaudioside D. In some embodiments, the steviol glycoside obtained comprises about 1% to about 5% Rebaudioside I on a dry weight basis. In one embodiment, the steviol glycoside obtained comprises about 4% Rebaudioside I. In some embodiments, the steviol glycoside obtained comprises about 21% to about 78% Rebaudioside M on a dry weight basis. In one embodiment, the steviol glycoside obtained comprises about 60% Rebaudioside M. In some embodiments, the steviol glycoside obtained comprises about 0% Stevioside and Rubusoside.

In further embodiments, the steviol glycosides obtained using the methods of the disclosure comprise Rebaudioside A, Rebaudioside D, Rebaudioside E, Rebaudioside I, and/or Rebaudioside M. Again, such embodiments are typical in accordance with the first or second mode of the disclosure, or the third mode where the stevia material comprises rubusoside, or the third mode where the rhamnosyl-containing steviol glycoside or the xylosyl-containing steviol glycoside is a minor component of the stevia material. In particular, such embodiments are typical where the stevia material comprises rebaudioside A and/or stevioside and/or rubusoside. In some embodiments, the steviol glycoside obtained comprises about 0% to about 2% Rebaudioside A on a dry weight basis. In one embodiment, the steviol glycoside obtained comprises about 1% Rebaudioside A. In some embodiments, the steviol glycoside obtained comprises about 0% to about 13% Rebaudioside D on a dry weight basis. In one embodiment, the steviol glycoside obtained comprises about 3% Rebaudioside D. In some embodiments, the steviol glycoside obtained comprises about 0% to about 10% Rebaudioside E on a dry weight basis. In one embodiment, the steviol glycoside obtained comprises about 6% Rebaudioside E. In some embodiments, the steviol glycoside obtained comprises about 0% to about 5%

Rebaudioside I on a dry weight basis. In one embodiment, the steviol glycoside obtained comprises about 4% Rebaudioside I. In some embodiments, the steviol glycoside obtained comprises about 65% to about 90% Rebaudioside M on a dry weight basis. In one embodiment, the steviol glycoside obtained comprises about 86% Rebaudioside M.

In yet further embodiments, the steviol glycosides obtained using the methods of the disclosure comprise Rebaudioside A, Rebaudioside D, Rebaudioside E, Rebaudioside I, and/or Rebaudioside M. Again, such embodiments are typical in accordance with the first or second mode of the disclosure, or the third mode where the stevia material comprises rubusoside, or the third mode where the rhamnosyl-containing steviol glycoside or the xylosyl-containing steviol glycoside is a minor component of the stevia material. In particular, such embodiments are typical where the stevia material comprises rebaudioside A and/or stevioside and/or rubusoside. In some embodiments, the steviol glycoside obtained comprises about 0% to about 2% Rebaudioside A on a dry weight basis. In some embodiments, the steviol glycoside obtained comprises about 1% Rebaudioside A. In some embodiments, the steviol glycoside obtained comprises about 0% to about 17% Rebaudioside D on a dry weight basis. In one embodiment, the steviol glycoside obtained comprises about 4% Rebaudioside D. In some embodiments, the steviol glycoside obtained comprises about 0% to about 13% Rebaudioside E on a dry weight basis. In one embodiment, the steviol glycoside obtained comprises about 1% Rebaudioside E. In some embodiments, the steviol glycoside obtained comprises about 1% to about 4% Rebaudioside I on a dry weight basis. In one embodiment, the steviol glycoside obtained comprises about 3% Rebaudioside I. In some embodiments, the steviol glycoside obtained comprises about 60% to about 80% Rebaudioside M on a dry weight basis. In one embodiment, the steviol glycoside obtained comprises about 75% Rebaudioside M.

Where the stevia material comprises rebaudioside A and/or stevioside and/or rubusoside, typically the one or more steviol glycosides obtained comprise rebaudioside M and at least one further steviol glycoside selected from rebaudioside D, rebaudioside E and rebaudioside I. More typically, the one or more steviol glycosides obtained comprise rebaudioside M and at least two further steviol glycosides selected from rebaudioside D, rebaudioside E and rebaudioside I.

In one embodiment, where the stevia material comprises rebaudioside A and/or stevioside and/or rubusoside, the one or more steviol glycosides obtained comprise rebaudioside M and rebaudioside D, optionally rebaudioside E and optionally rebaudioside I.

Where the stevia material comprises rebaudioside C, typically the one or more steviol glycosides obtained comprise at least one steviol glycoside selected from rebaudioside K, rebaudioside N2 and rebaudioside H4. For example, where the stevia material comprises (i) rebaudioside A and/or stevioside, and (ii) rebaudioside C as a minor component of the stevia material, typically the one or more steviol glycosides obtained comprise rebaudioside M, at least one further steviol glycoside selected from rebaudioside D, rebaudioside E and rebaudioside I, and at least one further steviol glycoside selected from rebaudioside K, rebaudioside N2 and rebaudioside H4. More typically, where the stevia material comprises (i) rebaudioside A and/or stevioside, and (ii) rebaudioside C as a minor component of the stevia material, the one or more steviol glycosides obtained comprise rebaudioside M, at least two further steviol glycosides selected from rebaudioside D, rebaudioside E and rebaudioside I, and at least two further steviol glycoside selected from rebaudioside K, rebaudioside N2 and rebaudioside H4.

Where the stevia material comprises rebaudioside C as a minor component of the stevia material, typically the combined rebaudioside K, rebaudioside N2 and rebaudioside H4 content is from 0.01 wt % to 50 wt % of the total steviol glycosides obtained. More typically in such an embodiment, the combined rebaudioside K, rebaudioside N2 and rebaudioside H4 content is from is from 0.1 wt % to 25 wt % of the total steviol glycosides obtained. Yet more typically in such an embodiment, the combined rebaudioside K, rebaudioside N2 and rebaudioside H4 content is from 0.5 wt % to 10 wt %. More typically still, the combined rebaudioside K, rebaudioside N2 and rebaudioside H4 content is from 1 wt % to 5 wt %.

Similarly, where the stevia material comprises rebaudioside F, typically the one or more steviol glycosides obtained comprise at least one β1-2 and/or β1-3 glucosylated derivative of rebaudioside F. For example, where the stevia material comprises (i) rebaudioside A and/or stevioside, and (ii) rebaudioside F as a minor component of the stevia material, typically the one or more steviol glycosides obtained comprise rebaudioside M, at least one further steviol glycoside selected from rebaudioside D, rebaudioside E and rebaudioside I, and at least one β1-2 and/or β1-3 glucosylated derivative of rebaudioside F. More typically, where the stevia material comprises (i) rebaudioside A and/or stevioside, and (ii) rebaudioside F as a minor component of the stevia material, the one or more steviol glycosides obtained comprise rebaudioside M, at least two further steviol glycosides selected from rebaudioside D, rebaudioside E and rebaudioside I, and at least two β1-2 and/or β1-3 glucosylated derivatives of rebaudioside F.

Where the stevia material comprises rebaudioside F as a minor component of the stevia material, typically the combined content of β1-2 and/or β1-3 glucosylated derivatives of rebaudioside F is from 0.01 wt % to 50 wt % of the total steviol glycosides obtained. More typically in such an embodiment, the combined content of β1-2 and/or β1-3 glucosylated derivatives of rebaudioside F is from 0.1 wt % to 15 wt % of the total steviol glycosides obtained. Yet more typically, the combined content of β1-2 and/or β1-3 glucosylated derivatives of rebaudioside F is from 0.5 wt % to 10 wt %. More typically still, the combined content of β1-2 and/or β1-3 glucosylated derivatives of rebaudioside F is from 1 wt % to 5 wt %.

In any embodiment where the steviol glycosides obtained comprise rebaudioside M and at least one further steviol glycoside selected from rebaudioside D, rebaudioside E and rebaudioside I, typically the rebaudioside M content is from 10 to 99 wt % of the total steviol glycosides obtained. More typically, the rebaudioside M content is from 20 to 95 wt % of the total steviol glycosides obtained. More typically still, the rebaudioside M content is from 40 to 90 wt %, or from 50 to 85 wt %, of the total steviol glycosides obtained. Yet more typically, the rebaudioside M content is from 60 to 80 wt % of the total steviol glycosides obtained.

Where the steviol glycosides obtained comprise rebaudioside D, typically the rebaudioside D content is from 0.1 to 99 wt % of the total steviol glycosides obtained. More typically, the rebaudioside D content is from 0.5 to 40 wt % of the total steviol glycosides obtained. Yet more typically, the rebaudioside D content is from 2 to 20 wt % of the total steviol glycosides obtained. More typically still, the rebaudioside D content is from 5 to 15 wt % of the total steviol glycosides obtained.

In certain embodiments, where the steviol glycosides obtained comprise rebaudioside M and rebaudioside D, the rebaudioside M content is from 56 to 85 wt % of the total steviol glycosides obtained and the rebaudioside D content is from 2 to 19 wt % of the total steviol glycosides obtained. Typically in such embodiments, the rebaudioside M content is from 58 to 82 wt % of the total steviol glycosides obtained and the rebaudioside D content is from 3 to 17 wt %. More typically in such embodiments, the rebaudioside M content is from 60 to 80 wt % and the rebaudioside D content is from 5 to 15 wt %.

In any embodiment where the steviol glycosides obtained comprise rebaudioside E, typically the rebaudioside E content is from 0.5 to 30 wt % of the total steviol glycosides obtained. More typically, the rebaudioside E content is from 2 to 20 wt % of the total steviol glycosides obtained. More typically still, the rebaudioside E content is from 5 to 15 wt % of the total steviol glycosides obtained.

In any embodiment where the steviol glycosides obtained comprise rebaudioside I, typically the rebaudioside I content is from 0.1 to 99 wt % of the total steviol glycosides obtained. More typically, the rebaudioside I content is from 0.5 to 25 wt % of the total steviol glycosides obtained. Yet more typically, the rebaudioside I content is from 1 to 10 wt % of the total steviol glycosides obtained. More typically still, the rebaudioside I content is from 1.5 to 5 wt % of the total steviol glycosides obtained.

In some embodiments, the steviol glycosides obtained comprise rebaudioside A. The rebaudioside A may be present either as a glycosylation product, or as residual unreacted rebaudioside A from the stevia material. For example, the rebaudioside A content may be from 0.1 to 50 wt % of the total steviol glycosides obtained. More typically in such embodiments, the rebaudioside A content is from 0.5 to 25 wt % of the total steviol glycosides obtained. More typically still, the rebaudioside A content is from 1 to 15 wt % of the total steviol glycosides obtained.

Alternately, the steviol glycosides obtained may comprise substantially no rebaudioside

A. For example, the rebaudioside A content may be <1 wt % of the total steviol glycosides obtained. More typically in such embodiments, the rebaudioside A content is <0.1 wt % or <0.01 wt % of the total steviol glycosides obtained.

Typically, the steviol glycosides obtained comprise substantially no stevioside. For example, the stevioside content may be <1 wt % of the total steviol glycosides obtained. More typically, the stevioside content is <0.1 wt % or <0.01 wt % of the total steviol glycosides obtained.

Typically, the steviol glycosides obtained comprise substantially no rubusoside. For example, the rubusoside content may be <1 wt % of the total steviol glycosides obtained. More typically, the rubusoside content is <0.1 wt % or <0.01 wt % of the total steviol glycosides obtained.

Typically, the total steviol glycosides obtained are obtained as part of a product, wherein the product has a total steviol glycoside content of ≥50% on a dry weight basis. Typically, the product has a total steviol glycoside content of ≥60% on a dry weight basis. More typically, the product has a total steviol glycoside content of ≥75% on a dry weight basis. For example, the product may have a total steviol glycoside content of ≥85% or ≥95% on a dry weight basis.

In some embodiments, the amount of steviol glycosides obtained using the methods of the disclosure is at least about 30% higher than the amount of steviol glycosides in the stevia material, on a dry weight basis. In further embodiments, the amount of steviol glycosides obtained is at least about 100% higher than the amount of steviol glycosides in the stevia material.

In some embodiments, the amount of steviol glycosides obtained using the methods of the disclosure is at least about 38% higher than the amount of steviol glycosides in the extract, on a dry weight basis. In further embodiments, the amount of steviol glycosides obtained is at least about 100% higher than the amount of steviol glycosides in the extract.

In some embodiments, the amount of steviol glycosides obtained using the methods of the disclosure is at least about 35% higher than the amount of steviol glycosides in the mother liquor, on a dry weight basis. In further embodiments, the amount of steviol glycosides is at least about 100% higher than the amount of steviol glycosides in the mother liquor.

In some embodiments, the steviol glycoside obtained using the methods of the disclosure comprises about 60% to about 95% minimally processed stevia material.

In some embodiments, the enzyme mixture improves mass gain efficiency by 14% to 100% based on Rebaudioside D to Rebaudioside M and/or Rubusoside to Rebaudioside M conversion.

In some embodiments, the steviol glycoside obtained using the methods of the disclosure is purified. In some embodiments, the purified steviol glycoside comprises Rebaudioside A, Rebaudioside D, Rebaudioside M, Rebaudioside E, Rebaudioside I, Stevioside, or Rubusoside. As will be appreciated, the methods of the disclosure may aid downstream purification techniques, not least because the steviol glycosides obtained may be separated from impurities and from each other more readily than the steviol glycosides in the source stevia material.

In some embodiments, the disclosure provides a method for obtaining a mixture of one or more steviol glycosides, the method comprising the steps of adding an enzyme mixture to a stevia material during an extraction process, wherein the stevia material is an extract, co-product, or waste product; incubating the enzyme mixture and stevia material; and obtaining a mixture of one or more steviol glycosides from the enzyme mixture and stevia material.

The method of the present disclosure may be used to catalyze the conversion of raw stevia material to various steviol glycosides. In some embodiments, the method is used to obtain a mixture of steviol glycosides that comprise Rebaudioside M, Rebaudioside A, and Rebaudioside D. In some embodiments the method is used to obtain a mixture of steviol glycosides comprising greater than 50% Rebaudioside M on a dry weight basis.

The mixtures of one or more steviol glycosides produced by the methods of the disclosure may comprise Rebaudioside M, Rebaudioside A, Rebaudioside D, or combinations thereof. In some embodiments, the mixture comprises Rebaudioside M, Rebaudioside A, Rebaudioside D, Rebaudioside I, and Rebaudioside E. In some embodiments, the mixture comprises at least 50% Rebaudioside M on a dry weight basis. In some embodiments, the mixture comprises at least 59% Rebaudioside M and at least one other steviol glycoside. In some embodiments, the mixture comprises at least 80% Rebaudioside M and at least one other steviol glycoside. In some embodiments, the mixture comprises at least 15% Rebaudioside A on a dry weight basis. In some embodiments, the mixture comprises at least 12% Rebaudioside A. In some embodiments, the mixture comprises at least 15% Rebaudioside D on a dry weight basis. In some embodiments, the mixture comprises at least 11% Rebaudioside D. In some embodiments, the mixture comprises about 5% Rebaudioside I on a dry weight basis. In some embodiments, the mixture comprises less than 1% Rebaudioside E. In some embodiments, the mixture comprises at least 94% Rebaudioside M on a dry weight basis and at least one other steviol glycoside. In some embodiments, the mixture comprises about 1% Rebaudioside I on a dry weight basis. In some embodiments, the mixture comprises less than 1% Rebaudioside A, Rebaudioside D, or Rebaudioside E on a dry weight basis. In some embodiments, the mixture comprises at least 86% Rebaudioside M on a dry weight basis and at least one other steviol glycoside. In some embodiments, the mixture comprises about 6% Rebaudioside E on a dry weight basis. In some embodiments, the mixture comprises about 4% Rebaudioside I on a dry weight basis. In some embodiments, the mixture comprises about 3% Rebaudioside I. In some embodiments, the mixture comprises less than 1% Rebaudioside A on a dry weight basis.

As stated above, the method of first aspect of the disclosure includes the step of incubating the enzyme mixture and the stevia material. Typical incubation conditions are set out below and are equally applicable to each mode of the first aspect of the disclosure.

The enzyme mixture and the stevia material are typically incubated in a solvent. Step (a) may therefore comprise adding the solvent to the stevia material. The solvent may be residual solvent from an earlier processing or extraction step, may be fresh solvent, or may be a combination of residual and fresh solvent. Typically the solvent is an alcoholic or an aqueous solvent. For example, the solvent may be water, methanol, ethanol, propanol, or a mixture thereof. More typically, the solvent is water, ethanol, or a mixture thereof. Most typically, the solvent is water.

Where the enzyme mixture and the stevia material are incubated in a solvent such as water, typically the stevia material is present at a concentration of from 0.1 to 300 g/L. Typically, the stevia material is present at a concentration of from 1 to 250 g/L. More typically, the stevia material is present at a concentration of from 25 to 200 g/L.

As stated above, the enzyme mixture comprises one or more glycosyltransferase enzymes. Typically, the enzyme mixture comprises one or more glucosyltransferase enzymes, such as one or more β1-2 and/or β1-3 glucosyltransferase enzymes (i.e. glucosyltransferase enzymes that are capable of forming β1-2 and/or β1-3 glucosyl linkages).

In one embodiment, the enzyme mixture comprises one or more nucleoside diphosphate (NDP)-dependent glycosyltransferases. For example, the enzyme mixture may comprise one or more NDP-glucose dependent glycosyltransferases, such as a one or more adenosine diphosphate (ADP)-glucose dependent, cytidine diphosphate (CDP)-glucose dependent, guanosine diphosphate (GDP)-glucose dependent, thymidine diphosphate (TDP)-glucose dependent, uridine diphosphate (UDP)-glucose dependent, or inosine diphosphate (IDP)-glucose dependent glycosyltransferases. Suitable examples of such enzymes that may be used in accordance with the present disclosure are disclosed in US 2020/0032227 A1. Typically, the enzyme mixture comprises one or more ADP-glucose dependent glycosyltransferases.

Typically, where the enzyme mixture comprises one or more NDP-glucose dependent glycosyltransferases, the enzyme mixture comprises one or more NDP-glucose dependent β1-2 and/or β1-3 glucosyltransferase enzymes. For example, the enzyme mixture may comprise one or more ADP-glucose dependent β1-2 and/or β1-3 glucosyltransferase enzymes.

Where the enzyme mixture and the stevia material are incubated in a solvent such as water, in some embodiments the one or more glycosyltransferase enzymes are present at a total concentration of from 0.01 to 10 g/L. Typically, the one or more glycosyltransferase enzymes are present at a total concentration of from 0.02 to 5 g/L. More typically, the one or more glycosyltransferase enzymes are present at a total concentration of from 0.05 to 2 g/L. Where the enzyme mixture comprises one or more glucosyltransferase enzymes, typically the enzyme mixture and the stevia material are incubated in the presence of a glucose source. For example, where the enzyme mixture comprises one or more NDP-glucose dependent glycosyltransferases, typically the enzyme mixture and the stevia material are incubated in the presence of a NDP-glucose source. Step (a) may therefore comprise adding the glucose source or the NDP-glucose source to the stevia material. As will be understood, where the enzyme mixture and the stevia material are incubated in the presence of a NDP-glucose source, the NDP-glucose source will be appropriate to the type of NDP-glucose dependent glycosyltransferase present. Thus, for example, where the enzyme mixture comprises one or more ADP-glucose dependent glycosyltransferases, typically the enzyme mixture and the stevia material are incubated in the presence of an ADP-glucose source; where the enzyme mixture comprises one or more CDP-glucose dependent glycosyltransferases, typically the enzyme mixture and the stevia material are incubated in the presence of a CDP-glucose source; where the enzyme mixture comprises one or more GDP-glucose dependent glycosyltransferases, typically the enzyme mixture and the stevia material are incubated in the presence of a GDP-glucose source; where the enzyme mixture comprises one or more TDP-glucose dependent glycosyltransferases, typically the enzyme mixture and the stevia material are incubated in the presence of a TDP-glucose source; where the enzyme mixture comprises one or more UDP-glucose dependent glycosyltransferases, typically the enzyme mixture and the stevia material are incubated in the presence of a UDP-glucose source; and where the enzyme mixture comprises one or more IDP-glucose dependent glycosyltransferases, typically the enzyme mixture and the stevia material are incubated in the presence of an IDP-glucose source.

In one embodiment, the NDP-glucose source is a NDP-glucose. Step (a) may therefore comprise adding the NDP-glucose to the stevia material. For example, the NDP-glucose source may be ADP-glucose, CDP-glucose, GDP-glucose, TDP-glucose, UDP-glucose or IDP-glucose.

Where the enzyme mixture and the stevia material are incubated in a solvent such as water, and the NDP-glucose source is a NDP-glucose, typically the NDP-glucose is present at a concentration of from 0.1 to 200 g/L. More typically, the NDP-glucose is present at a concentration of from 1 to 100 g/L. More typically still, the NDP-glucose is present at a concentration of from 25 to 75 g/L.

In another embodiment, the NDP-glucose is generated in situ. For example, in one embodiment, the enzyme mixture further comprises a sucrose synthase. Typically in such an embodiment, the enzyme mixture and the stevia material are incubated in the presence of sucrose and a nucleoside diphosphate (NDP), such as ADP, CDP, GDP, TDP, UDP or IDP (depending on the type of NDP-glucose dependent glycosyltransferase present). Step (a) may therefore comprise adding the NDP and the sucrose to the stevia material. Suitable examples of sucrose synthases that may be used in accordance with the present disclosure are disclosed in US 2020/0032227 A1.

Where the enzyme mixture comprises a sucrose synthase and the enzyme mixture and the stevia material are incubated in a solvent such as water, typically the sucrose synthase is present at a concentration of from 0.01 to 10 g/L. More typically, the sucrose synthase is present at a concentration of from 0.02 to 2 g/L. More typically still, the sucrose synthase is present at a concentration of from 0.05 to 0.5 g/L.

Typically, where the enzyme mixture comprises a sucrose synthase and the enzyme mixture and the stevia material are incubated in a solvent such as water, sucrose is present at a concentration of from 0.1 to 500 g/L. More typically, sucrose is present at a concentration of from 1 to 300 g/L. More typically still, sucrose is present at a concentration of from 50 to 250 g/L.

Typically, where the enzyme mixture comprises a sucrose synthase and the enzyme mixture and the stevia material are incubated in a solvent such as water, the NDP is present at a concentration of from 0.01 to 10 g/L. More typically, the NDP is present at a concentration of from 0.05 to 1 g/L. More typically still, the NDP is present at a concentration of from 0.1 to 0.5 g/L.

Optionally, the incubation step is performed in the presence of a chelating agent such as EDTA. Step (a) may therefore comprise adding the chelating agent to the stevia material. Typically, where the enzyme mixture and the stevia material are incubated in a solvent such as water, the chelating agent is present at a concentration of from 0.01 to 10 g/L. More typically, the chelating agent is present at a concentration of from 0.02 to 5 g/L. More typically still, the chelating agent is present at a concentration of from 0.05 to 2 g/L.

Where the enzyme mixture and the stevia material are incubated in a solvent such as water, typically the incubation occurs at a pH of from 5 to 9. More typically the incubation occurs at a pH of from 5.5 to 8.5. More typically still, the incubation occurs at a pH of from 6.0 to 8.0. Optionally, a buffer may be used in order to maintain the desired pH. In one embodiment, the buffer is selected from potassium phosphate or tris(hydroxymethyl)aminomethane hydrochloride (Tris-HCl).

In one embodiment, the incubation occurs at a temperature of from 20 °C to 90 °C. Typically, the incubation occurs at a temperature of from 40 °C to 85 °C. More typically, the incubation occurs at a temperature of from 50 °C to 80 °C.

In some embodiments, the enzyme mixture and stevia material are incubated for at least 2 hours. In further embodiments, the enzyme mixture and stevia material are incubated for between 4 hours and 8 hours. In yet further embodiments, the enzyme mixture and extract are incubated for between 6 hours and 24 hours.

In some embodiments, the enzyme mixture and mother liquor are incubated for at least 6 hours. In further embodiments, the enzyme mixture and mother liquor are incubated for between 14 hours and 30 hours.

Optionally, the method of the first aspect of the disclosure further comprises the step of deactivating the enzyme mixture at the end of the incubation period. In one embodiment said deactivation step comprises heat treating the incubation mixture to deactivate the enzyme mixture. For example, the incubation mixture may be heated to a temperature of at least 90 °C, e.g. to a temperature in the range from 90-110 °C, or 95-100 °C. Alternately or in addition, the deactivation step may comprise quenching the incubation mixture with a suitable acid such as formic or acetic acid.

The residual deactivated enzyme may optionally be removed by filtration, centrifugation, or other suitable separation technique.

Optionally, the method of the first aspect of the disclosure further comprises the step of removing the solvent at the end of the incubation period, typically after or simultaneously with the step of deactivating the enzyme mixture.

Optionally, the method of the first aspect of the disclosure further comprises the step of purifying the one or more steviol glycosides obtained to obtain a purified composition. Any conventional purification technique known to those skilled in the art may be employed, including but not limited to solvent evaporation, drying, precipitation, (re) crystallization, filtration, centrifugation, chromatography, biphasic solvent washes, treatment with ion exchange resin(s), treatment with activated carbon, and any combination thereof.

In some embodiments, the purification step results in a solid purified composition. In other embodiments, the purification step results in a liquid purified composition, for example a solution of one or more steviol glycosides.

Typically the purification step ensures that the total steviol glycoside content of the purified composition is ≥95% on a dry weight basis. In some embodiments, the total steviol glycoside content of the purified composition is ≥98% or ≥99% on a dry weight basis.

Where the one or more steviol glycosides obtained comprise a mixture of steviol glycosides, in some embodiments the purified composition is a purified mixture of steviol glycosides.

In other embodiments, the purified composition comprises ≥95% of a single steviol glycoside, such as rebaudioside M, rebaudioside D or rebaudioside I, on a dry weight basis. Typically in such an embodiment, the purified composition comprises ≥98% or ≥99% of a single steviol glycoside on a dry weight basis.

In a particular embodiment, the purified composition comprises ≥95% of rebaudioside M on a dry weight basis. Typically in such an embodiment, the purified composition comprises ≥98% or ≥99% of rebaudioside M on a dry weight basis.

### Products of the Enzymatic Conversion

As stated above, according to a second aspect of the disclosure, there is provided a product comprising one or more steviol glycosides, wherein the product is obtained or obtainable by a method of the first aspect of the disclosure. In some embodiments, the product is the crude product obtained or obtainable at the end of the incubation step, optionally after the step of deactivating the enzyme mixture and/or removing the solvent. In other embodiments, the product is a purified composition as set out above.

In some embodiments, the disclosure provides a product comprising one or more steviol glycosides produced by adding an enzyme mixture to a stevia material during an extraction process, wherein the stevia material is an extract, co-product, or waste product; incubating the enzyme mixture and stevia material; and obtaining one or more steviol glycosides from the enzyme mixture and stevia material.

The product comprising one or more steviol glycosides produced by the methods of the present disclosure may comprise, on a dry weight basis, a mixture of 7-15% Rebaudioside A, 6
14% Rebaudioside D, 0.2-4% Rebaudioside E, 1-9% Rebaudioside I, 49-66% Rebaudioside M, and the remainder is stevia extract. In another embodiment, the product comprises, on a dry weight basis, a mixture of 0.2-3% Rebaudioside A, 1-8% Rebaudioside D, 3-10% Rebaudioside E, 1-8% Rebaudioside I, 74-94% Rebaudioside M, and the remainder is stevia extract. In another embodiment, the product comprises, on a dry weight basis, a mixture of 0.2-3% Rebaudioside A, 0.2-7% Rebaudioside D, 0.2-3% Rebaudioside E, 0.2-8% Rebaudioside I, 68-97% Rebaudioside M, and the remainder is mother liquor or stevia extract.

In one embodiment of the second aspect of the disclosure, the product comprises rebaudioside M and at least one further steviol glycoside selected from rebaudioside D, rebaudioside E and rebaudioside I. More typically, the product comprises rebaudioside M and at least two further steviol glycosides selected from rebaudioside D, rebaudioside E and rebaudioside I. For example, the product may comprise rebaudioside M and rebaudioside D, optionally rebaudioside E and optionally rebaudioside I.

In any embodiment where the product comprises rebaudioside M and at least one further steviol glycoside selected from rebaudioside D, rebaudioside E and rebaudioside I, typically the rebaudioside M content is from 10 to 99 wt % of the total steviol glycoside content of the product. More typically, the rebaudioside M content is from 20 to 95 wt % of the total steviol glycoside content of the product. More typically still, the rebaudioside M content is from 40 to 90 wt %, or from 50 to 85 wt %, of the total steviol glycoside content of the product. Yet more typically, the rebaudioside M content is from 60 to 80 wt % of the total steviol glycoside content of the product.

Where the product comprises rebaudioside D, typically the rebaudioside D content is from 0.1 to 99 wt % of the total steviol glycoside content of the product. More typically the rebaudioside D content is from 0.5 to 40 wt % of the total steviol glycoside content of the product. Yet more typically, the rebaudioside D content is from 2 to 20 wt % of the total steviol glycoside content of the product. More typically still, the rebaudioside D content is from 5 to 15 wt % of the total steviol glycoside content of the product.

According to a third aspect of the disclosure, there is provided a composition comprising rebaudioside M and rebaudioside D, wherein the rebaudioside M content is from 56 wt % to 85 wt % of the total steviol glycoside content of the composition, and wherein the rebaudioside D content is from 2 wt % to 19 wt % of the total steviol glycoside content of the composition.

Typically in accordance with the third aspect of the disclosure, the rebaudioside M content is from 58 wt % to 82 wt % of the total steviol glycoside content of the composition. More typically, the rebaudioside M content is from 60 wt % to 80 wt % of the total steviol glycoside content of the composition. Typically, the rebaudioside D content is from 3 wt % to 17 wt % of the total steviol glycoside content of the composition. More typically, the rebaudioside D content is from 5 wt % to 15 wt % of the total steviol glycoside content of the composition.

In one embodiment of the third aspect of the disclosure, the composition further comprises rebaudioside E and/or rebaudioside I.

Typically, the composition of the third aspect of the disclosure is obtained or obtainable by a method of the first aspect of the disclosure. For example, the composition may be obtained or obtainable by a method of the first or the second mode of the first aspect of the disclosure, or by a method of the third mode of the first aspect of the disclosure where the stevia material comprises rubusoside.

According to a fourth aspect of the disclosure, there is provided a composition comprising rebaudioside M and at least one further steviol glycoside selected from rebaudioside K, rebaudioside N2 and/or rebaudioside H4. Typically, said composition is obtained or obtainable by a method of the third mode of the first aspect of the disclosure where the stevia material comprises rebaudioside A, stevioside, and as a minor component rebaudioside C.

Typically the rebaudioside M content is from 10 to 99 wt % of the total steviol glycoside content of the composition of the fourth aspect of the disclosure. More typically, the rebaudioside M content is from 20 to 95 wt % of the total steviol glycoside content of the composition. More typically still, the rebaudioside M content is from 40 to 90 wt %, or from 50 to 85 wt %, of the total steviol glycoside content of the composition. Yet more typically, the rebaudioside M content is from 60 to 80 wt % of the total steviol glycoside content of the composition.

Typically the combined rebaudioside K, rebaudioside N2 and rebaudioside H4 content is from 0.01 wt % to 50 wt % of the total steviol glycoside content of the composition of the fourth aspect of the disclosure. More typically, the combined rebaudioside K, rebaudioside N2 and rebaudioside H4 content is from 0.1 wt % to 25 wt % of the total steviol glycoside content of the composition. Yet more typically, the combined rebaudioside K, rebaudioside N2 and rebaudioside H4 content is from 0.5 wt % to 10 wt % of the total steviol glycoside content of the composition. More typically still, the combined rebaudioside K, rebaudioside N2 and rebaudioside H4 content is from 1 wt % to 5 wt % of the total steviol glycoside content of the composition.

According to a fifth aspect of the disclosure, there is provided a composition comprising rebaudioside M and at least one β1-2 and/or β1-3 glucosylated derivative of rebaudioside F. Typically, said composition is obtained or obtainable by a method of the third mode of the first aspect of the disclosure where the stevia material comprises rebaudioside A, stevioside, and as a minor component rebaudioside F.

Typically the rebaudioside M content is from 10 to 99 wt % of the total steviol glycoside content of the composition of the fifth aspect of the disclosure. More typically, the rebaudioside M content is from 20 to 95 wt % of the total steviol glycoside content of the composition. More typically still, the rebaudioside M content is from 40 to 90 wt %, or from 50 to 85 wt %, of the total steviol glycoside content of the composition. Yet more typically, the rebaudioside M content is from 60 to 80 wt % of the total steviol glycoside content of the composition. Typically the combined content of β1-2 and/or β1-3 glucosylated derivatives of rebaudioside F is from 0.01 wt % to 50 wt % of the total steviol glycoside content of the composition of the fifth aspect of the disclosure. More typically, combined content of β1-2 and/or β1-3 glucosylated derivatives of rebaudioside F is from 0.1 wt % to 15 wt % of the total steviol glycoside content of the composition. Yet more typically, combined content of β1-2 and/or β1-3 glucosylated derivatives of rebaudioside F is from 0.5 wt % to 10 wt % of the total steviol glycoside content of the composition. More typically still, the combined content of β1-2 and/or β1-3 glucosylated derivatives of rebaudioside F is from 1 wt % to 5 wt % of the total steviol glycoside content of the composition.

In one embodiment equally applicable to both the fourth and the fifth aspects of the disclosure, the composition further comprises at least one further steviol glycoside selected from rebaudioside D, rebaudioside E and rebaudioside I. More typically, the composition further comprises at least two further steviol glycosides selected from rebaudioside D, rebaudioside E and rebaudioside I. For example, the composition may further comprise rebaudioside D, optionally rebaudioside E and optionally rebaudioside I.

Where the composition of the fourth or fifth aspect of the disclosure comprises rebaudioside D, typically the rebaudioside D content is from 0.1 to 99 wt % of the total steviol glycoside content of the composition. More typically the rebaudioside D content is from 0.5 to 40 wt % of the total steviol glycoside content of the composition. Yet more typically, the rebaudioside D content is from 2 to 20 wt % of the total steviol glycoside content of the composition. More typically still, the rebaudioside D content is from 5 to 15 wt % of the total steviol glycoside content of the composition.

Where the product or composition of any of the second to fifth aspects of the disclosure comprises rebaudioside E, typically the rebaudioside E content is from 0.5 to 30 wt % of the total steviol glycoside content of the product or composition. More typically, the rebaudioside E content is from 2 to 20 wt % of the total steviol glycoside content of the product or composition. More typically still, the rebaudioside E content is from 5 to 15 wt % of the total steviol glycoside content of the product or composition.

Where the product or composition of any of the second to fifth aspects of the disclosure comprises rebaudioside I, typically the rebaudioside I content is from 0.1 to 99 wt % of the total steviol glycoside content of the product or composition. More typically, the rebaudioside I content is from 0.5 to 25 wt % of the total steviol glycoside content of the product or composition. Yet more typically, the rebaudioside I content is from 1 to 10 wt % of the total steviol glycoside content of the product or composition. More typically still, the rebaudioside I content is from 1.5 to 5 wt % of the total steviol glycoside content of the product or composition.

In some embodiments the product or composition of any of the second to fifth aspects of the disclosure comprises rebaudioside A. The rebaudioside A may be present either as a glycosylation product, or as residual unreacted rebaudioside A from the stevia material. For example, the rebaudioside A content may be from 0.1 to 50 wt % of the total steviol glycoside content of the product or composition. More typically in such embodiments, the rebaudioside A content is from 0.5 to 25 wt % of the total steviol glycoside content of the product or composition. More typically still, the rebaudioside A content is from 1 to 15 wt % of the total steviol glycoside content of the product or composition.

Alternately, the product or composition of any of the second to fifth aspects of the disclosure may comprise substantially no rebaudioside A. For example, the rebaudioside A content may be <1 wt % of the total steviol glycoside content of the product or composition. More typically in such embodiments, the rebaudioside A content is <0.1 wt % or <0.01 wt % of the total steviol glycoside content of the product or composition.

Typically, the product or composition of any of the second to fifth aspects of the disclosure comprises substantially no stevioside. For example, the stevioside content may be <1 wt % of the total steviol glycoside content of the product or composition. More typically, the stevioside content is <0.1 wt % or <0.01 wt % of the total steviol glycoside content of the product or composition.

Typically, the product or composition of any of the second to fifth aspects of the disclosure comprises substantially no rubusoside. For example, the rubusoside content may be <1 wt % of the total steviol glycoside content of the product or composition. More typically, the rubusoside content is <0.1 wt % or <0.01 wt % of the total steviol glycoside content of the product or composition.

Typically, the product or composition of any of the second to fifth aspects of the disclosure has a total steviol glycoside content of ≥50% on a dry weight basis. More typically, the product or composition has a total steviol glycoside content of ≥60% on a dry weight basis. More typically still, the product or composition has a total steviol glycoside content of ≥75% on a dry weight basis. For example, the product or composition may have a total steviol glycoside content of ≥85% or ≥95% on a dry weight basis.

In some embodiments of any of the second to fifth aspects of the disclosure, the product or composition is a solid. In other embodiments, the product or composition is a liquid, typically an aqueous solution.

Typically in accordance with any of the second to fifth aspects of the disclosure, the product or composition is suitable for human consumption. Typically in accordance with any of the second to seventh aspects of the disclosure, the product or composition is suitable for use as a sweetener.

### Applications

The products and compositions produced by the methods of the present disclosure may be blended with further components that are suitable for human and/or animal consumption, to produce sweetener compositions, foodstuffs, beverages and the like. The one or more steviol glycosides produced by the methods of the present disclosure may be at least partially purified prior to use in a product.

Accordingly, a sixth aspect of the disclosure provides a method of preparing a sweetener composition, the method comprising the steps of obtaining one or more steviol glycosides by the method of the first aspect of the present disclosure, and blending the steviol glycosides obtained with one or more co-sweeteners. Similarly, a seventh aspect of the disclosure provides a method of preparing a sweetener composition, the method comprising the step of blending a product or composition of any of the second to fifth aspects of the disclosure with one or more co-sweeteners. A corresponding eighth aspect of the disclosure provides a sweetener composition, wherein the sweetener composition comprises a product or composition of any of the second to fifth aspects of the disclosure, and one or more co-sweeteners. Typically in accordance with the sixth, seventh and eighth aspects of the disclosure, the one or more co-sweeteners are selected from the group consisting of stevia extracts, steviol glycosides, glycosylated steviol glycosides, monk fruit extracts, mogrosides, glycosylated mogrosides, sucrose, glucose, fructose, high-fructose corn syrup, sucralose, saccharin, acesulfame K, sodium cyclamate, aspartame, neotame and advantame. In certain embodiments, the one or more co-sweeteners are selected from the groups consisting of monk fruit extracts, mogrosides, glycosylated mogrosides, sucrose, glucose, fructose, high-fructose corn syrup, sucralose, saccharin, acesulfame K, sodium cyclamate, aspartame, neotame and advantame. Typically in accordance with the sixth aspect of the disclosure, the steviol glycosides obtained are blended with the one or more co-sweeteners at a dry weight ratio of from 5:95 to 95:5. Typically in accordance with the seventh and eighth aspects of the disclosure, the dry weight ratio of the one or more co-sweeteners to the product or composition of any of the second to fifth aspects of the disclosure is from 5:95 to 95:5.

In some embodiments, the product comprising one or more steviol glycosides produced by the methods of the present disclosure is a consumable product that may be used for human and/or animal consumption. In some embodiments, the consumable product is a food product, a beverage product, a pharmaceutical product, a nutritional product, or a nutraceutical.

A ninth aspect of the disclosure provides a method of preparing a food product or a beverage product, the method comprising the steps of obtaining one or more steviol glycosides by the method of the first aspect of the present disclosure and blending the steviol glycosides obtained with the food product, the beverage product, or an ingredient thereof. Similarly, a tenth aspect of the disclosure provides a method of preparing a food product or a beverage product, the method comprising the step of blending a product or composition of any of the second to fifth aspects of the disclosure with the food product, the beverage product, or an ingredient thereof. A corresponding eleventh aspect of the disclosure provides a food product or a beverage product, wherein the food product or the beverage product comprises a product or composition of any of the second to fifth aspects of the disclosure.

A twelfth aspect of the disclosure provides a method of preparing a food product or a beverage product, the method comprising the steps of preparing a sweetener composition by the method of the sixth or the seventh aspect of the present disclosure and blending the sweetener composition with the food product, the beverage product, or an ingredient thereof. Similarly, a thirteenth aspect of the disclosure provides a method of preparing a food product or a beverage product, the method comprising the step of blending the sweetener composition of the eighth aspect of the present disclosure with the food product, the beverage product, or an ingredient thereof. A corresponding fourteenth aspect of the disclosure provides a food product or a beverage product, wherein the food product or the beverage product comprises a sweetener composition of the eighth aspect of the present disclosure.

Typically, the resultant food product or beverage product of any of the ninth to fourteenth aspects of the disclosure comprises steviol glycosides in an amount from 0.0001 to 1% by weight.

In some embodiments, when the consumable product is a food product, the food product is a confectionary product, a dessert product, a cereal product, a baked good, a frozen dairy product, a meat, a dairy product, a condiment, a snack bar, a nutritional bar, a soup, a dressing, a mix, a prepared food, a baby food, a diet preparation, a syrup, a food coating, a dried fruit, a sauce, a gravy, or a jam or jelly.

In some embodiments, when the consumable product is a beverage product, the beverage product is an alcoholic beverage, a carbonated soft drink, a fruit juice, a tea, or a smoothie. A fifteenth aspect of the disclosure provides a method of preparing a pharmaceutical product, a nutritional product, or a nutraceutical, the method comprising the steps of obtaining one or more steviol glycosides by the method of the first aspect of the present disclosure and blending the steviol glycosides obtained with the pharmaceutical product, the nutritional product, the nutraceutical, or a precursor thereof. Similarly, a sixteenth aspect of the disclosure provides a method of preparing a pharmaceutical product, a nutritional product, or a nutraceutical, the method comprising the step of blending a product or composition of any of the second to fifth aspects of the disclosure with the pharmaceutical product, the nutritional product, the nutraceutical, or a precursor thereof. A corresponding seventeenth aspect of the disclosure provides a pharmaceutical product, a nutritional product, or a nutraceutical, wherein the pharmaceutical product, the nutritional product or the nutraceutical comprises a product or composition of any of the second to fifth aspects of the disclosure.

An eighteenth aspect of the disclosure provides a method of preparing a pharmaceutical product, a nutritional product, or a nutraceutical, the method comprising the steps of preparing a sweetener composition by the method of the sixth or the seventh aspect of the present disclosure and blending the sweetener composition with the pharmaceutical product, the nutritional product, the nutraceutical, or a precursor thereof. Similarly, a nineteenth aspect of the disclosure provides a method of preparing a pharmaceutical product, a nutritional product, or a nutraceutical, the method comprising the step of blending the sweetener composition of the eighth aspect of the present disclosure with the pharmaceutical product, the nutritional product, the nutraceutical, or a precursor thereof. A corresponding twentieth aspect of the disclosure provides a pharmaceutical product, a nutritional product, or a nutraceutical, wherein the pharmaceutical product, the nutritional product, or the nutraceutical comprises a sweetener composition of the eighth aspect of the present disclosure.

Typically, the resultant pharmaceutical product, nutritional product, or nutraceutical of any of the fifteenth to twentieth aspects of the disclosure comprises steviol glycosides in an amount from 0.0001 to 5% by weight.

In some embodiments, the product is a nutraceutical, and the nutraceutical is selected from the group consisting of cannabidiol products (CBD), cannabis-derived product, hemp-derived products, and the like.

Various exemplary embodiments of compositions and methods according to this disclosure are now described in the following non-limiting Examples. The Examples are offered for illustrative purposes only and are not intended to limit the scope of the present disclosure in any way. Indeed, various modifications of the disclosure in addition to those shown and described herein will become apparent to those skilled in the art from the foregoing description and the following Examples and fall within the scope of the appended claims.

### EXAMPLES

The Examples that follow are illustrative of specific embodiments of the disclosure, and various uses thereof. They are set forth for explanatory purposes only and should not be construed as limiting the scope of the disclosure in any way.

In each enzyme incubation step below, an aqueous solution of the stevia material (e.g. the early extraction product or the mother liquor product), buffered to a pH of 6.5, was incubated at a temperature of 65 °C for the stated time period in the presence of an ADP-glucose dependent glucosyltransferase enzyme (at a concentrations of 0.1 and 0.4 g/L), sucrose synthase (at a concentration of 0.1 g/L), sucrose (at a concentration of 150 g/L), ADP (at a concentration of 0.1 to 0.5 g/L) and EDTA (at a concentration of 1.9 g/L).

### Example 1: Early Stevia Extraction Product and Reaction

### 1.0 Hours

An early extraction product from stevia manufacture contains the glycosides detailed in Table 1. The early extraction product is a minimally processed stevia extract that contains 60-95% total steviol glycoside on a dry weight basis. In this example, the early extraction product contained 83.8% total steviol glycoside content on a dry weight basis.

TABLE 1

| | wt % of total steviol glycoside content |
|---|---|
| Rebaudioside A | 56.34% |
| Rebaudioside D | 2.57% |
| Rebaudioside I | 0% |
| Rebaudioside M | 0.32% |
| Stevioside | 28.61% |
| Rubusoside | 0.48% |

### 2. 2, 5 and 7 Hours

This early extraction product is dissolved in water to produce a solution having a total steviol glycoside content of 8.03 g/100 mL. After 2, 5 and 7 hours of reaction with enzymes the early extraction product from Table 1 was converted to the glycosides detailed in Table 2. Due to the increase in the molecular weights of the steviol glycosides during enzymatic bioconversion, there is a mass gain of the stevia components. After 2, 5, and 7 hours of bioconversion there are approximately 18, 27, and 31% increases in steviol glycoside mass post reaction compared to the original extract.

TABLE 2

| | wt % of total | wt % of total | wt % of total |
|---|---|---|---|
| | steviol | steviol | steviol |
| | glycoside | glycoside | glycoside |
| | content | content | content |
| | after 2 hours | after 5 hours | after 7 hours |
| Rebaudioside A | 40.82% | 19.82% | 11.92% |
| Rebaudioside D | 18.78% | 15.63% | 13.61% |
| Rebaudioside I | 3.41% | 4.40% | 4.71% |
| Rebaudioside M | 25.86% | 53.29% | 62.38% |
| Stevioside | 1.45% | 0% | 0% |
| Rubusoside | 0% | 0% | 0% |
| % mass increase | 18% | 27% | 31% |
| of total steviol | | | |
| glycoside content | | | |

### Example 2: Mother Liquor Product and Reaction

### 1. 0 Hours

Mother liquor is a co-product stream obtained from crystallization of a stevia stream that contains 40-95% total steviol glycoside on a dry weight basis. In this example, a batch of mother liquor that contained 77.8% total steviol glycosides on a dry weight basis was dissolved in water to produce a solution having a total Reb M convertible content of 4.89 g/100 mL. The solution contained the steviol glycosides detailed in Table 3.

TABLE 3

| | wt % of Reb M Convertibles* |
|---|---|
| Rebaudioside A | 44.30% |
| Rebaudioside D | 4.37% |
| Rebaudioside M | 2.03% |
| Rebaudioside E | 0.92% |
| Rebaudioside I | 0% |
| Stevioside | 46.56% |
| Rubusoside | 1.82% |

*wt % of Reb M Convertibles = the amount of each stated steviol glycoside as a wt % of the total rebaudioside A + rebaudioside D + rebaudioside M + rebaudioside E + rebaudioside I + stevioside + rubusoside content.

### 2. 14 Hours

After 14 hours of reaction with enzymes the mother liquor product from Table 3 was converted to the glycosides detailed in Table 4. Due to the mass gain associated with this reaction there is an approximate 37% increase in steviol glycosides post reaction compared to the original mother liquor.

TABLE 4

| | wt % of Reb M Convertibles |
|---|---|
| Rebaudioside A | 1.06% |
| Rebaudioside D | 3.99% |
| Rebaudioside M | 77.58% |
| Rebaudioside E | 13.64% |
| Rebaudioside I | 3.73% |
| Stevioside | 0% |
| Rubusoside | 0% |

### Example 3: Purification of Early Stevia Extraction Product

The end product from the reaction presented in Example 1 can be purified by carbon treatment, adsorption desorption resins and dried with an expected yield of 108% from the original steviol glycoside content. This may be further purified by solvent washing to achieve a 90% yield from the original steviol glycosides.

### Example 4: Purification of Mother Liquor Product

Material from the reaction as presented in Example 2 can be purified by filtration and dried with an expected yield of 104% from the original steviol glycoside content.

### Example 5: Glycosylation

Glycosylation of Rebaudioside C was conducted using ADP, sucrose synthase, sucrose and one or both of β1-2 and β1-3 ADP-glucose dependent glycosyltransferases. After exposure to enzymatic reaction conditions for 24 hours, Rebaudioside C was nearly fully converted to Rebaudioside K or Rebaudioside N2. However, conversion to Rebaudioside H4 was relatively slow. Conversion of Rebaudioside C to a 5 glucosyl residues and 1 rhamnosyl containing derivative represents a 34.1% mass gain.

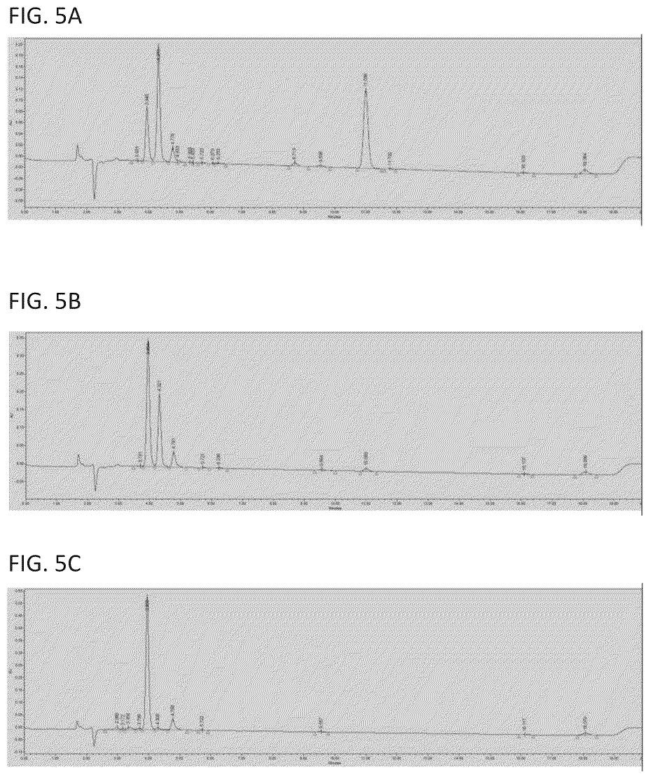

The steviol glycosides in the materials presented in FIG. 5A-5C include a steviol glycoside with 1 xylosyl and four glucosyl group linked to the steviol core (i.e., Rebaudioside F). The products from this reaction show glycosylated xylose containing steviol glycosides in LC-MS. Three products formed contain 5 glucosyl residues and 1 xylosyl residue. This shows that there are multiple xylosyl substitution patterns, with glucosyl filling in the other available 2 and 3 positions of the glucosyl group directly linked to steviol. Conversion of Rebaudioside F to a 5 glucosyl residues and 1 xylosyl containing derivative represents a 34.6% mass gain.

### Example 6: Mother Liquor Product and Reaction

1. 0 hours

A batch of mother liquor that contained 62.3% total steviol glycosides on a dry weight basis was dissolved in water to produce to produce a solution having a total steviol glycoside content of 8.02 g/100 mL. The solution contained the steviol glycosides detailed in Table 5.

TABLE 5

| | wt % of total steviol glycoside content |
|---|---|
| Rebaudioside A | 38.14% |
| Rebaudioside D | 4.3% |
| Rebaudioside M | 5.91% |
| Rebaudioside I | 0.26% |
| Stevioside | 16.24% |
| Rubusoside | 1.75% |

2. 27 hours

After 27 hours of reaction with enzymes the mother liquor product from Table 4 was converted to the glycosides detailed in Table 6.

TABLE 6

| | wt % of total steviol glycoside content |
|---|---|
| Rebaudioside A | 0% |
| Rebaudioside D | 8.51% |
| Rebaudioside M | 71.63% |
| Rebaudioside I | 1.93% |
| Stevioside | 0% |
| Rubusoside | 0% |

### Example 7: Purification of Mother Liquor Product

Material from the reaction as presented in Example 6 can be purified by filtration and dried to produce a product containing 92.68% Reb M and 101.96% total steviol glycosides on a dry weight basis.

### Example 8: Organoleptic Evaluation of the Isolated Product of Example 1

The material from example 1, after 7 hours was isolated and dried to produce a product containing 12.4% Reb A, 13.01% Reb D, 58.8% Reb M, and 96.4% total steviol glycosides, all on dry weight basis. Organoleptic evaluation of this material was compared against commercial RA60-TSG95 and RA97 samples. The results are shown in Table 7. Samples were prepared in neutral water at 500 ppm concentrations. Eight trained panelists evaluated these solutions for sweet taste intensity, off-flavor and liking. Sweet taste intensity was compared to sucrose standards (sucrose equivalence value, SEV), and other attributes on a scale from 0-7.

TABLE 7

| | ppm | SEV | liking | off-flavor |
|---|---|---|---|---|
| Enzyme converted product | 500 | 9.8 | 4.3 | 1.6 |
| Rebaudioside A 60 | 500 | 6.6 | 3.6 | 2.4 |
| Rebaudioside A 97 | 500 | 7.7 | 3.5 | 2.8 |

As can be seen, at a concentration of 500 μm, the product prepared in accordance with the present disclosure is able to produce superior sweetness and likeability and have less off-flavour than commercially available products.

### CLAUSES OF THE INVENTION

Clause 1: A method for obtaining one or more steviol glycosides, the method comprising the steps of:
   (a) adding an enzyme mixture to a stevia material during an extraction process, wherein the stevia material is an extract or co-product, or waste product;
   (b) incubating the enzyme mixture and stevia material; and
   (c) obtaining one or more steviol glycoside from the enzyme mixture and stevia material.
Clause 2: The method of clause 1, wherein the enzyme mixture comprises a glycosyltransferase and a sucrose synthase.
Clause 3: The method of clause 1, wherein the stevia material comprises an extract.
Clause 4: The method of clause 1, wherein the stevia material comprises a waste product.
Clause 5: The method of clause 4, wherein the waste product comprises a mother liquor co-product stream obtained from crystallization of a stevia stream.
Clause 6: The method of clause 5, wherein the mother liquor comprises about 40% to about 95% steviol glycoside on a dry weight basis.
Clause 7: The method of clause 1, wherein the steviol glycoside comprises one or more of Rebaudioside A, Rebaudioside D, Rebaudioside M, Rebaudioside E, Rebaudioside I, Stevioside, and/or Rubusoside.
Clause 8: The method of clause 7, wherein the steviol glycoside comprises Rebaudioside A,
Clause 9: The method of clause 8, wherein the steviol glycoside comprises about 1% to about 38% Rebaudioside A.
Clause 10: The method of clause 9, wherein the steviol glycoside comprises about 11% Rebaudioside A.
Clause 11: The method of clause 8, wherein the steviol glycoside comprises about 10% to about 33% Rebaudioside D.
Clause 12: The method of clause 11, wherein the steviol glycoside comprises about 13% Rebaudioside D.
Clause 13: The method of clause 8, wherein the steviol glycoside comprises about 1% to about 5% Rebaudioside I.
Clause 14: The method of clause 13, wherein the steviol glycoside comprises about 4% Rebaudioside I.
Clause 15: The method of clause 8, wherein the steviol glycoside comprises about 21% to about 78% Rebaudioside M.
Clause 16: The method of clause 15, wherein the steviol glycoside comprises about 60% Rebaudioside M.
Clause 17: The method of clause 1, wherein the steviol glycoside comprises about 0% Stevioside and Rubusoside.
Clause 18: The method of clause 3, wherein the steviol glycoside comprises Rebaudioside A,
Clause 19: The method of clause 18, wherein the steviol glycoside comprises about 0% to about 2% Rebaudioside A.
Clause 20: The method of clause 19, wherein the steviol glycoside comprises about 1% Rebaudioside A.
Clause 21: The method of clause 18, wherein the steviol glycoside comprises about 0% to about 13% Rebaudioside D.
Clause 22: The method of clause 21, wherein the steviol glycoside comprises about 3% Rebaudioside D.
Clause 23: The method of clause 18, wherein the steviol glycoside comprises about 0% to about 10% Rebaudioside E.
Clause 24: The method of clause 23, wherein the steviol glycoside comprises about 6% Rebaudioside E.
Clause 25: The method of clause 18, wherein the steviol glycoside comprises about 0% to about 5% Rebaudioside I.
Clause 26: The method of clause 25, wherein the steviol glycoside comprises about 4% Rebaudioside I.
Clause 27: The method of clause 18, wherein the steviol glycoside comprises about 65% to about 90% Rebaudioside M.
Clause 28: The method of clause 27, wherein the steviol glycoside comprises about 86% Rebaudioside M.
Clause 29: The method of clause 5, wherein the steviol glycoside comprises Rebaudioside A, Rebaudioside D, Rebaudioside E, Rebaudioside I, and/or Rebaudioside M.
Clause 30: The method of clause 29, wherein the steviol glycoside comprises about 0% to about 2% Rebaudioside A.
Clause 31: The method of clause 30, wherein the steviol glycoside comprises about 1% Rebaudioside A.
Clause 32: The method of clause 29, wherein the steviol glycoside comprises about 0% to about 17% Rebaudioside D.
Clause 33: The method of clause 32, wherein the steviol glycoside comprises about 4% Rebaudioside D.
Clause 34: The method of clause 29, wherein the steviol glycoside comprises about 0% to about 13% Rebaudioside E.
Clause 35: The method of clause 34, wherein the steviol glycoside comprises about 1% Rebaudioside E.
Clause 36: The method of clause 29, wherein the steviol glycoside comprises about 1% to about 4% Rebaudioside I.
Clause 37: The method of clause 36, wherein the steviol glycoside comprises about 3% Rebaudioside I.
Clause 38: The method of clause 29, wherein the steviol glycoside comprises about 60% to about 80% Rebaudioside M.
Clause 39: The method of clause 38, wherein the steviol glycoside comprises about 75% Rebaudioside M.
Clause 40: The method of clause 1, wherein the enzyme mixture and stevia material are incubated for at least 2 hours.
Clause 41: The method of clause 40, wherein the enzyme mixture and stevia material are incubated for between 4 hours and 8 hours.
Clause 42: The method of clause 3, wherein the enzyme mixture and extract are incubated for between 4 hours and 24 hours.
Clause 43: The method of clause 5, wherein the enzyme mixture and mother liquor are incubated for at least 6 hours.
Clause 44: The method of clause 43, wherein the enzyme mixture and mother liquor are incubated for between 14 hours and 30 hours.
Clause 45: The method of clause 1, wherein the amount of steviol glycoside is at least about 30% higher than the amount of steviol glycoside in the stevia material.
Clause 46: The method of clause 45, wherein the amount of steviol glycoside is at least about 100% higher than the amount of steviol glycoside in the stevia material.
Clause 47: The method of clause 3, wherein the amount of steviol glycoside is at least about 38% higher than the amount of steviol glycoside in the extract.
Clause 48: The method of clause 47, wherein the amount of steviol glycoside is at least 100% higher than the amount of steviol glycoside in the extract.
Clause 49: The method of clause 5, wherein the amount of steviol glycoside is at least about 35% higher than the amount of steviol glycoside in the mother liquor.
Clause 50: The method of clause 49, wherein the amount of steviol glycoside is at least about 100% higher than the amount of steviol glycoside in the mother liquor.
Clause 51: The method of clause 1, wherein the steviol glycoside comprises about 60% to about 95% minimally processed stevia material.
Clause 52: The method of clause 1, wherein the enzyme mixture improves mass gain efficiency by 14% to 100% based on Rebaudioside D to Rebaudioside M and/or Rubusoside to Rebaudioside M conversion.
Clause 53: The method of clause 1, further comprising purifying the steviol glycoside.
Clause 54: The method of clause 53, wherein at least 85% of the purified steviol glycoside comprises Rebaudioside A, Rebaudioside D, Rebaudioside M, Rebaudioside E, Rebaudioside I, Stevioside, or Rubusoside.
Clause 55: A method for obtaining a mixture of one or more steviol glycosides, the method comprising the steps of:
   (a) adding an enzyme mixture to a stevia material during an extraction process, wherein the stevia material is an extract, co-product, or waste product;
   (b) incubating the enzyme mixture and stevia material; and
   (c) obtaining a mixture of one or more steviol glycosides from the enzyme mixture and stevia material.
Clause 56: A mixture of one or more steviol glycosides produced by the method of clause 55, wherein the mixture comprises Rebaudioside M, Rebaudioside A, Rebaudioside D, or combinations thereof.
Clause 57: The mixture of clause 56, wherein the mixture comprises at least 50% Rebaudioside M.
Clause 58: The mixture of clause 56, wherein the mixture comprises at least 80% Rebaudioside M and at least one other steviol glycoside.
Clause 59: The mixture of clause 56, wherein the mixture comprises at least 15% Rebaudioside A.
Clause 61: The mixture of clause 56, wherein the mixture comprises at least 15% Rebaudioside D.
Clause 62: A method for obtaining a purified mixture of one or more steviol glycosides, the method comprising the steps of:
   (a) adding an enzyme mixture to a stevia material during an extraction process, wherein the stevia material is an extract, co-product, or waste product;
   (b) incubating the enzyme mixture and stevia material;
   (c) obtaining a mixture of one or more steviol glycosides from the enzyme mixture and stevia material;
   (d) purifying the mixture of the one or more steviol glycosides.
Clause 63: A purified mixture of one or more steviol glycosides produced by the method of clause 62, wherein the mixture comprises Rebaudioside M, Rebaudioside A, Rebaudioside D, Rebaudioside E, Rebaudioside I, or combinations thereof.
Clause 64: The purified mixture of clause 63, wherein the purified mixture comprises at least 50% Rebaudioside M.
Clause 65: The purified mixture of clause 64, wherein the purified mixture comprises at least 50% Rebaudioside M and at least one other steviol glycoside.
Clause 66: The purified mixture of clause 63, wherein the purified mixture comprises at least 10% Rebaudioside A.
Clause 67: The purified mixture of clause 63, wherein the purified mixture comprises at least 10% Rebaudioside D.
Clause 68: The purified mixture of clause 64, wherein the purified mixture comprises at least 70% Rebaudioside M.
Clause 69: The purified mixture of clause 68, wherein the purified mixture comprises at least 90% Rebaudioside M and at least one other steviol glycoside.
Clause 70: The purified mixture of clause 65, wherein the at least one other steviol glycoside comprises less than 5% of the mixture.
Clause 71: A product comprising one or more steviol glycosides produced by:
   (a) adding an enzyme mixture to a stevia material during an extraction process, wherein the stevia material is an extract or waste product;
   (b) incubating the enzyme mixture and stevia material; and
   (c) obtaining one or more steviol glycosides from the enzyme mixture and stevia material.
Clause 72: The product of clause 71, wherein the product is a consumable product.
Clause 73: The consumable product of clause 72, wherein the consumable product is a food product, a beverage product, a pharmaceutical product, a nutritional product, or a nutraceutical product.
Clause 74: The consumable product of clause 73, wherein the food product is a confectionery product, a dessert product, a cereal product, a baked good, a frozen dairy product, a meat product, a dairy product, a condiment, a snack bar, a nutritional bar, a soup, a dressing, a mix, a prepared food, a baby food, a diet preparation, a syrup, a food coating or frosting, a dried fruit, a sauce, a gravy, or a jam or jelly.
Clause 75: The beverage product of clause 73, wherein the beverage product is an alcoholic beverage, a carbonated soft drink, a fruit juice, a tea, or a smoothie.
Clause 76: The nutraceutical product of clause 73, wherein the nutraceutical product is a cannabis or cannabis-derived product.
Clause 77: The product of clause 71, wherein the one or more steviol glycosides are at least partially purified.
Clause 78: The product of clause 71, where in the one or more steviol glycosides comprise a mixture of 7-15% Rebaudioside A, 6-14% Rebaudioside D, 0.2-4% Rebaudioside E, 1-9% Rebaudioside I, 49-66% Rebaudioside M, and the remainder is stevia extract.
Clause 79: The product of clause 71, where in the one or more steviol glycosides comprise a mixture of 0.2-3% Rebaudioside A, 1-8% Rebaudioside D, 3-10% Rebaudioside E, 1-8% Rebaudioside I, 74-94% Rebaudioside M, and the remainder is stevia extract.
Clause 80: The product of clause 71, where in the one or more steviol glycosides comprise a mixture of 0.2-3% Rebaudioside A, 0.2-7% Rebaudioside D, 0.2-3% Rebaudioside E, 0.2-8% Rebaudioside I, 68-97% Rebaudioside M, and the remainder is mother liquor.

1.-46. (canceled)

47. A method for obtaining one or more steviol glycosides, the method comprising the steps of:
(a) adding an enzyme mixture to a stevia material;
(b) incubating the enzyme mixture and stevia material; and
(c) obtaining the one or more steviol glycosides from the enzyme mixture and stevia material;
wherein the enzyme mixture comprises one or more nucleoside diphosphate-glucose dependent glycosyltransferases.

48. The method as claimed in claim 47, wherein the stevia material has a total steviol glycoside content of:
(i) ≤90% on a dry weight basis; or
(ii) from 60% to 85% on a dry weight basis.

49. The method as claimed in claim 48, wherein the stevia material comprises rebaudioside A, stevioside, optionally one or more additional steviol glycosides selected from the group consisting of rebaudioside D, rebaudioside E, rebaudioside I, rebaudioside M and rubusoside, and optionally one or more further steviol glycosides, and optionally wherein:
(i) the one or more steviol glycosides obtained comprise rebaudioside M and at least one further steviol glycoside selected from rebaudioside D, rebaudioside E and rebaudioside I; and/or
(ii) the total content of rebaudioside A and stevioside in the stevia material is from 50 to 95 wt % of the total steviol glycoside content of the stevia material.

50. The method as claimed in claim 47, wherein the stevia material comprises rebaudioside A, stevioside and rebaudioside D, and optionally wherein:
(i) the stevia material further comprises one or more additional steviol glycosides selected from the group consisting of rebaudioside E, rebaudioside I, rebaudioside M and rubusoside, and optionally one or more further steviol glycosides; and/or
(ii) the one or more steviol glycosides obtained comprise rebaudioside M and at least one further steviol glycoside selected from rebaudioside D, rebaudioside E and rebaudioside I; and/or
(iii) the total content of rebaudioside A and stevioside in the stevia material is from 50 to 95 wt % of the total steviol glycoside content of the stevia material; and/or
(iv) the rebaudioside A content is from 20 to 80 wt % of the total steviol glycoside content of the stevia material, the stevioside content is from 10 to 60 wt % of the total steviol glycoside content of the stevia material, and the rebaudioside D content is from 0.5 to 15 wt % of the total steviol glycoside content of the stevia material; and/or
(v) the rebaudioside A content is from 35 to 60 wt % of the total steviol glycoside content of the stevia material, the stevioside content is from 15 to 50 wt % of the total steviol glycoside content of the stevia material, and the rebaudioside D content is from 2 to 5 wt % of the total steviol glycoside content of the stevia material.

51. The method as claimed in claim 48, wherein:
(i) the stevia material comprises rebaudioside E, and the rebaudioside E content is from 0.1 to 5 wt % of the total steviol glycoside content of the stevia material; and/or
(ii) the stevia material comprises rebaudioside I, and the rebaudioside I content is from 0.05 to 2 wt % of the total steviol glycoside content of the stevia material; and/or
(iii) the stevia material comprises rebaudioside M, and the rebaudioside M content is from 0.1 to 15 wt % of the total steviol glycoside content of the stevia material; and/or
(iv) the stevia material comprises rubusoside, and the rubusoside content is from 0.1 to 5 wt % of the total steviol glycoside content of the stevia material.

52. The method as claimed in claim 47, wherein the stevia material comprises rubusoside and/or a rhamnosyl-containing steviol glycoside and/or a xylosyl-containing steviol glycoside, and optionally wherein:
(i) the stevia material comprises rubusoside and the one or more steviol glycosides obtained comprise rebaudioside M and at least one further steviol glycoside selected from rebaudioside D, rebaudioside E and rebaudioside I; and/or
(ii) the stevia material comprises rebaudioside C and the one or more steviol glycosides obtained comprise at least one steviol glycoside selected from rebaudioside K, rebaudioside N2 and rebaudioside H4; and/or
(iii) the stevia material comprises rebaudioside F and the one or more steviol glycosides obtained comprise at least one β1-2 and/or β1-3 glucosylated derivative of rebaudioside F.

53. The method as claimed in claim 52, wherein:
(i) the stevia material contains ≥75% rubusoside on a dry weight basis; or
(ii) the stevia material contains ≥75% of the rhamnosyl-containing steviol glycoside on a dry weight basis; or
(iii) the stevia material contains ≥75% of the xylosyl-containing steviol glycoside on a dry weight basis.

54. The method as claimed in claim 52, wherein:
(i) the stevia material has a rubusoside content of from 0.01 wt % to 10 wt % of the total steviol glycoside content of the stevia material; or
(ii) the stevia material has a rebaudioside C content of from 0.1 wt % to 25 wt % of the total steviol glycoside content of the stevia material; or
(iii) the stevia material has a rebaudioside F content of from 0.1 wt % to 15 wt % of the total steviol glycoside content of the stevia material.

55. The method as claimed in claim 54, wherein the stevia material further comprises rebaudioside A, stevioside, optionally one or more additional steviol glycosides selected from the group consisting of rebaudioside D, rebaudioside E, rebaudioside I, rebaudioside M and rubusoside, and optionally one or more further steviol glycosides, and optionally wherein:
(i) the one or more steviol glycosides obtained comprise rebaudioside M and at least one further steviol glycoside selected from rebaudioside D, rebaudioside E and rebaudioside I; and/or
(ii) the total content of rebaudioside A and stevioside in the stevia material is from 50 to 95 wt % of the total steviol glycoside content of the stevia material.

56. The method as claimed in claim 47, wherein:
(i) the stevia material is an extract, co-product, or waste product from a stevia extraction process; and/or
(ii) the enzyme mixture comprises one or more NDP-glucose dependent β1-2 and/or β1-3 glucosyltransferase enzymes; and/or
(iii) the enzyme mixture further comprises a sucrose synthase, and wherein the enzyme mixture and the stevia material are incubated in the presence of sucrose and a nucleoside diphosphate (NDP).

57. A product comprising one or more steviol glycosides, wherein the product is obtained or obtainable by the method as claimed in claim 47, and optionally wherein:
(i) the product comprises, on a dry weight basis, a mixture of 7-15% Rebaudioside A, 6-14% Rebaudioside D, 0.2-4% Rebaudioside E, 1-9% Rebaudioside I, 49-66% Rebaudioside M, and the remainder is stevia extract; or
(ii) the product comprises, on a dry weight basis, a mixture of 0.2-3% Rebaudioside A, 1-8% Rebaudioside D, 3-10% Rebaudioside E, 1-8% Rebaudioside I, 74-94% Rebaudioside M, and the remainder is stevia extract; or
(iii) the product comprises, on a dry weight basis, mixture of 0.2-3% Rebaudioside A, 0.2-7% Rebaudioside D, 0.2-3% Rebaudioside E, 0.2-8% Rebaudioside I, 68-97% Rebaudioside M, and the remainder is mother liquor or stevia extract.

58. A composition comprising rebaudioside M and rebaudioside D, wherein the rebaudioside M content is from 56 wt % to 85 wt % of the total steviol glycoside content of the composition, and wherein the rebaudioside D content is from 2 wt % to 19 wt % of the total steviol glycoside content of the composition; and optionally wherein:
(i) the rebaudioside M content is from 60 wt % to 80 wt % of the total steviol glycoside content of the composition, and wherein the rebaudioside D content is from 5 wt % to 15 wt % of the total steviol glycoside content of the composition; and/or
(ii) the composition further comprises rebaudioside E and/or rebaudioside I; and/or
(iii) the stevioside content is <0.1 wt % of the total steviol glycoside content of the composition and the rubusoside content is <0.1 wt % of the total steviol glycoside content of the composition.

59. A composition comprising rebaudioside M and optionally rebaudioside D, wherein the composition further comprises:
(i) at least one further steviol glycoside selected from rebaudioside K, rebaudioside N2 and rebaudioside H4, optionally wherein the rebaudioside M content is from 20 to 95 wt % of the total steviol glycoside content of the composition and the combined rebaudioside K, rebaudioside N2 and rebaudioside H4 content is from 0.1 wt % to 25 wt % of the total steviol glycoside content of the composition; or
(ii) at least one β1-2 and/or β1-3 glucosylated derivative of rebaudioside F, optionally wherein the rebaudioside M content is from 20 to 95 wt % of the total steviol glycoside content of the composition and the combined content of β1-2 and/or β1-3 glucosylated derivatives of rebaudioside F is from 0.1 wt % to 15 wt % of the total steviol glycoside content of the composition.

60. The composition as claimed in claim 59, wherein:
(i) the composition further comprises rebaudioside E and/or rebaudioside I; and/or
(ii) the stevioside content is <0.1 wt % of the total steviol glycoside content of the composition and the rubusoside content is <0.1 wt % of the total steviol glycoside content of the composition.

61. A method of preparing a sweetener composition, the method comprising the steps of obtaining one or more steviol glycosides by the method as claimed in claim 47, and blending the steviol glycosides obtained with one or more co-sweeteners.

62. A sweetener composition, food product, beverage product, pharmaceutical product, nutritional product or nutraceutical, wherein the sweetener composition, the food product, the beverage product, the pharmaceutical product, the nutritional product or the nutraceutical comprises a product as claimed in claim 57, and wherein the sweetener composition further comprises one or more co-sweeteners.

63. A method of preparing a food product or a beverage product, the method comprising the steps of obtaining one or more steviol glycosides by the method as claimed in claim 47, and blending the steviol glycosides obtained with the food product, the beverage product, or an ingredient thereof.

64. A sweetener composition, food product, beverage product, pharmaceutical product, nutritional product or nutraceutical, wherein the sweetener composition, the food product, the beverage product, the pharmaceutical product, the nutritional product or the nutraceutical comprises a composition as claimed in claim 58, and wherein the sweetener composition further comprises one or more co-sweeteners.

65. A method of preparing a pharmaceutical product, a nutritional product, or a nutraceutical, the method comprising the steps of obtaining one or more steviol glycosides by the method as claimed in claim 47, and blending the steviol glycosides obtained with the pharmaceutical product, the nutritional product, the nutraceutical, or a precursor thereof.

66. A sweetener composition, food product, beverage product, pharmaceutical product, nutritional product, or nutraceutical, wherein the sweetener composition, the food product, the beverage product, the pharmaceutical product, the nutritional product or the nutraceutical comprises a composition as claimed in claim 59, and wherein the sweetener composition further comprises one or more co-sweeteners.
